SEQUENCE LISTING (26 sequences)

SEQ ID NO: 1 - DNA, 19 bp, Homo sapiens
tgtacaagtgtcagctaag

SEQ ID NO: 2 - DNA, 21 bp, Homo sapiens
gaaccatgtggataactttac

SEQ ID NO: 3 - DNA, 25 bp, Homo sapiens
gcacgagctacctcagcaagacgtt

SEQ ID NO: 4 - DNA, 25 bp, Homo sapiens
cctgaagaaggcagagatatggcaa

SEQ ID NO: 5 - DNA, 25 bp, Homo sapiens
gaccaaagatggagtgagagaggtt

SEQ ID NO: 6 - DNA, 25 bp, Homo sapiens
gaattaggctgtaactactttataa

SEQ ID NO: 7 - DNA, 21 bp, Artificial Sequence
  source: /note=“Description of Artificial Sequence Synthetic oligonucleotide”
uguacaagugucagcuaagtt

SEQ ID NO: 8 - DNA, 21 bp, Artificial Sequence
  source: /note=“Description of Artificial Sequence Synthetic oligonucleotide”
cuuagcugacacuuguacatt

SEQ ID NO: 9 - DNA, 23 bp, Artificial Sequence
  source: /note=“Description of Artificial Sequence Synthetic oligonucleotide”
gaaccauguggauaacuuuactt

SEQ ID NO: 10 - DNA, 23 bp, Artificial Sequence
  source: /note=“Description of Artificial Sequence Synthetic oligonucleotide”
guaaaguuauccacaugguuctt

SEQ ID NO: 11 - DNA, 25 bp, Artificial Sequence
  source: /note=“Description of Artificial Sequence Synthetic oligonucleotide”
gcacgagcuaccucagcaagacgtt

SEQ ID NO: 12 - RNA, 27 nt, Artificial Sequence
  source: /note=“Description of Artificial Sequence Synthetic oligonucleotide”
aacgucuugcugagguagcucgugcug

SEQ ID NO: 13 - DNA, 25 bp, Artificial Sequence
  source: /note=“Description of Artificial Sequence Synthetic oligonucleotide”
ccugaagaaggcagagauauggcaa

SEQ ID NO: 14 - RNA, 27 nt, Artificial Sequence
  source: /note=“Description of Artificial Sequence Synthetic oligonucleotide”
uugccauaucucugccuucuucagguu

SEQ ID NO: 15 - DNA, 25 bp, Artificial Sequence
  source: /note=“Description of Artificial Sequence Synthetic oligonucleotide”
gaccaaagauggagugagagaggtt

SEQ ID NO: 16 - RNA, 27 nt, Artificial Sequence
  source: /note=“Description of Artificial Sequence Synthetic oligonucleotide”
aaccucucucacuccaucuuuggucuu

SEQ ID NO: 17 - DNA, 25 bp, Artificial Sequence
  source: /note=“Description of Artificial Sequence Synthetic oligonucleotide”
gaauuaggcuguaacuacuuuauaa

SEQ ID NO: 18 - RNA, 27 nt, Artificial Sequence
  source: /note=“Description of Artificial Sequence Synthetic oligonucleotide”
uuauaaaguaguuacagccuaauucac

SEQ ID NO: 19 - DNA, 20 bp, Artificial Sequence
  source: /note=“Description of Artificial Sequence Synthetic primer”
aaagaacctgccccagaaat

SEQ ID NO: 20 - DNA, 20 bp, Artificial Sequence
  source: /note=“Description of Artificial Sequence Synthetic primer”
tggtggtggaacttctttcc

SEQ ID NO: 21 - DNA, 20 bp, Artificial Sequence
  source: /note=“Description of Artificial Sequence Synthetic probe”
aatcctggaaaatgtgcctg

SEQ ID NO: 22 - DNA, 20 bp, Artificial Sequence
  source: /note=“Description of Artificial Sequence Synthetic primer”
tatcgaggtggatggaaagc

SEQ ID NO: 23 - DNA, 20 bp, Artificial Sequence
  source: /note=“Description of Artificial Sequence Synthetic primer”
ttctggggtccacttttctg

SEQ ID NO: 24 - DNA, 20 bp, Artificial Sequence
  source: /note=“Description of Artificial Sequence Synthetic probe”
ccatcgacagccctgatagt

SEQ ID NO: 25 - DNA, 25 bp, Artificial Sequence
  source: /note=“Description of Artificial Sequence Synthetic oligonucleotide”
gcgacguaaacggccacaagttcag

SEQ ID NO: 26 - RNA, 27 nt, Artificial Sequence
  source: /note=“Description of Artificial Sequence Synthetic oligonucleotide”
cugaacuuguggccguuuacgucgccg

(19) United States
(12) Patent Application Publication
(10) Pub. No.: US 2011/0293625 A1
(43) Pub. Date: Dec. 1, 2011

(54) INHIBITION OF VEGF-A SECRETION, ANGIOGENESIS AND/OR NEOANGIOGENESIS BY SINA MEDIATED KNOCKDOWN OF VEGF-C AND RHOA

(76) Inventors: Krishna Addepalli Murali, Maharashtra (IN); Kumar Bharat, Maharashtra (IN); Chile Shailaja, Maharashtra (IN)

(21) Appl. No.: 13/130,699

(22) PCT Filed: Nov. 20, 2009
(86) PCT No.: PCT/IN2009/000671

§ 371 Date: May 23, 2011

(30) Foreign Application Priority Data
Nov. 21, 2008 (IN) 2459/MUM/2008

(51) Int. Cl. A61K 39/395; A61K 31/7105; A61P 27/06; A61P 35/00; A61P 27/02; C12N 5/071; A61K 31/711

(52) U.S. Cl. 424/141.1; 435/375; 514/44 R; 514/44 A

(57) ABSTRACT

The invention relates to the use of short interfering nucleic acid molecules (siNAs, such as siRNAs) that modulate the expression of VEGF-C and/or RhoA involved in neovascular angiogenesis. In the present invention, inhibition of VEGF-C and/or RhoA gene expression lead to decreased expression of VEGF-A, which is required for initiation and the sustaining of angiogenesis. Further, the invention also relates to the inhibition of RhoA expression levels along with VEGF-C, so as to derive the benefits of down-regulating two different targets required for angiogenesis. The present invention describes compounds, compositions and methods useful for inhibition of neoangiogenesis. In certain embodiments, the invention relates to methods for inhibiting neovascularization, as well as compounds, such as VEGF-C and RhoA siRNAs, useful in the treatment of ocular disorders such as age related maculardegeneration (AMD), diabetic retinopathy, glaucoma and other neovascular disorders.

### SEQUENCE LISTING

The instant application contains a Sequence Listing which has been submitted via EFS-Web and is hereby incorporated by reference in its entirety. Said ASCII copy, created on Nov. 18, 2009, is named RLS PCT 037. txt, and is 7,300 bytes in size.

### CROSS-REFERENCE TO RELATED PATENT APPLICATIONS

This application claims benefit of provisional Indian Application No. 2459/MUM/2008, filed Nov. 21, 2008, which is hereby entirely incorporated by reference.

### FIELD OF THE INVENTION

The present invention relates to use of short nucleic acid molecules, such as short interfering nucleic acid (siNA) molecules, for modulating gene and protein expression, including compounds, compositions and synergistic combination of small nucleic acid molecules that modulate RhoA and/or VEGF-C gene expression. The compounds and methods of the present invention have applications in modulating Rho-A, VEGF-C and VEGF-A expression and secretion, angiogenesis and/or neoangiogenesis, either alone or in combination with other therapies.

### BACKGROUND OF THE INVENTION

### Angiogenesis

Angiogenesis is the formation of new blood vessels or enlargement of existing vessels, while lymphangiogenesis is the equivalent process in lymphatic vessels. The processes of blood and lymphatic angiogenesis are tightly regulated by several key angiogenic factors. Anti-angiogenic factors include thrombospondin,platelet factor IV, TNF-alpha (in vitro), TGF-beta, interferons, angiostatin, integrin inhibitors, 16 kD prolactin, endostatin, and ANG-2. Certain steroids may inhibit angiogenesis.

Proangiogenic factors for blood and lymph vessels include FGF (fibroblast growth factor), VEGF (vascular endothelial growth factor), PDGF (platelet-derived growth factor), and angiopoietin families.

The FGF family is known to have broad biological functions on a variety of cell types. FGF-1 and FGF-2 are potent angiogenic factors in vivo, although the physiological and pathological relevance of these factors in regulation of angiogenesis is unclear.

The VEGF family includes at least five structurally related proteins, VEGF-A, VEGF-B, VEGF-C, VEGF-D, and placenta growth factor (P1GF). These molecules interact with a set of cell surface receptors, VEGFR-1, VEGFR-2, and VEGFR-3, that show varying specificity and function. VEGF-C and VEGF-D bind to both VEGFR-2 and VEGFR-3 and promote formation of blood and lymph vessels. VEGF-B and P1GF bind to VEGFR-1 and modulate the effects of VEGF-A, but their roles in stimulation of angiogenesis remain controversial. In addition to its ability to stimulate angiogenesis, VEGF-A acts as a potent vascular permeability factor (VPF). A large body of work indicates that VEGFR-2 is the receptor that mediates VEGF-A-induced angiogenic and permeability effects. In support of this notion, VEGF-B and P1GF, which only interact with VEGFR-1, lack angiogenic and vascular permeability activity. VEGF-A, VEGF-C, and FGF-2 determine the thickness of the endothelial cell layer in capillaries (in order of importance, VEGF-A<VEGF-C<FGF-2). In contrast to blood capillaries, lymph capillaries generated by VEGF-C completely lack fenestrations. This may be due to differences in signaling pathways used for angiogenesis and lymphangiogenesis. Hyperglycemia, hypoxia or higher levels of insulin increases expression of VEGF-C in retinal endothelial as well as epithelial cells, and increased VEGF-C suppresses endothelial cell apoptosis.

Regulation of angiogenesis is critical for numerous normal and pathological processes, including embryonic development, wound healing, tumor growth and metastasis, rheumatoid arthritis, diabetic retinopathy, atherosclerosis, and revascularization of ischemic myocardium, hind limb muscles, and brain. Lymphangiogenesis is critically important for tumor spread via the lymphatic system.

Neovascularization has been shown to cause or exacerbate ocular diseases including, but not limited to, macular degeneration, neovascular glaucoma, diabetic retinopathy, myopic degeneration, and trachoma. Norrby APMIS 105, 417-437 (1997). It has been reported that the ocular fluid of a majority of patients suffering from diabetic retinopathy and other retinal disorders contains a high concentration of VEGF-A. Aiello et al. New Engl. J. Med. 331, 1480 (1994). Elevated levels of VEGF mRNA in patients suffering from retinal ischemia have also been reported. Miller et al., Am. J. Pathol. 145, 574 (1994). Thus, VEGF-A may have a direct role in ocular diseases. Other factors, including those that stimulate VEGF synthesis, may also contribute to these indications.

### RhoA

The Ras superfamily of proteins are involved in the regulation and timing of cell division, and include Rho. The Rho family of proteins includes RhoA, RhoB, RhoC, Rac1, Rac2 and Cdc42, which share more than 50% sequence identity with each other. Rho proteins are involved in inducing the formation of stress fibers and focal contacts in response to extracellular signals such as lysophosphatidic acid (LPA) and growth factors. Ridley et al. Cell, 70, 389-399 (1992); Ridley et al., EMBO J., 1353, 2600-2610 (1994). The Rho family is also considered to be implicated in physiological functions associated with cytoskeletal rearrangements, such as cell morphological change (Parterson et al., J. Cell Biol., 111, 1001-1007 (1990)), cell adhesion (Morii et al., J. Biol. Chem., 267, 20921-20926 (1992); Tominaga et al., J. Cell Biol., 120, 1529-1537 (1993); Nusrat et al., Proc. Natl. Acad. Sci. USA, 92, 10629-10633 (1995); Landanna et al., Science, 271, 981-983 (1996)), cell motility (Takaishi et al., Oncogene, 9, 273-279 (1994)), and cytokinesis. (Kishi et al., J. Cell Biol., 120, 1187-1195 (1993); I. Mabuchi et al., Zygote, 1, 325-331 (1993)). It has been suggested that Rho proteins are involved in the regulation of smooth muscle contraction (Hirata et al., J. Biol. Chem., 267, 8719-8722 (1992); Noda et al., FEBS Lett., 367, 246-250 (1995); Gong et al., Proc. Natl. Acad. Sci. USA, 93, 1340-1345 (1996)), and the expression of phosphatidylinositol 3-kinase (PI3 kinase) (Zhang et al., J. Biol. Chem., 268, 22251-22254 (1993)), phosphatidylinositol 4-phosphate 5-kinase (PI 4,5-kinase) (Chong et al., Cell, 79, 507-513 (1994)) and c-fos (Hill et al., Cell, 81, 1159-1170 (1995)).

RhoA is a small GTPase protein known to regulate the actin cytoskeleton in the formation of stress fibers. Rho-kinase (ROCK) proteins are downstream effectors of RhoA and are activated through phosphorylation by activated Rho. The mammalian ROCK family includes ROCK-1 and ROCK-2, which are serine/threonine kinases of about 160 kDa, encoded by two different genes.

### OBJECTS OF THE INVENTION

An object of the present invention is to provide a combination of short interfering nucleic acid (siNA) molecules for modulation of VEGF-C and/or RhoA gene expression that display better specificity and/or effectiveness than prior art short interfering nucleic acids directed against either protein.

It is an object of the present invention to modulate expression levels of VEGF-C, which affects cellular levels of VEGF-A.

It is an object of present invention to reduce the activity of the VEGFR-3 receptor by knocking down expression levels of VEGF-C, its interacting ligand.

It is an object of the present invention to disrupt the homeostasis between VEGF-C and VEGF-A to control neoangiogenesis.

It is an object of the present invention to inhibit (i.e., reduce or decrease) neoangiogenesis by decreasing expression levels of VEGF-C and/or RhoA genes.

It is an object of the present invention to inhibit neoangiogenesis by providing to a cell, vessel, tissue, or organism, siNAs that inhibit expression of VEGF-C and/or RhoA genes.

It is an object of the present invention to inhibit RhoA gene expression so that a VEGF-A gene or protein, or a gene or protein involved in a VEGF-A mediated signaling pathway, is inhibited or reduced in amount, thereby regulating neoangiogenesis.

It is an object of the present invention to inhibit VEGF-C gene expression so that a VEGF-A gene or protein, or a gene or protein involved in a VEGF-A mediated signaling pathway, is inhibited or reduced in amount, thereby regulating neoangiogenesis.

It is an object of the present invention to inhibit RhoA and VEGF-C gene expression so that a VEGF-A gene or protein, or a gene or protein involved in a VEGF-A mediated signaling pathway, is inhibited or reduced in amount, for example, synergistically, thereby regulating neoangiogenesis.

It is an object of the present invention to provide RhoA and VEGF-C siNAs in combination to a cell, vessel, tissue or organism, whereby VEGF-A expression and secretion, angiogenesis and/or neoangiogenesis is inhibited in a synergistic manner, as compared to what is observed when adding the effects of using each of RhoA and/or VEGF-C siNA individually.

It is an object of the present invention to provide compounds comprising 27-mer siNA molecules that inhibit gene expression of RhoA and/or VEGF-C.

It is an object of the present invention to provide siNAs comprising about 19 to about 30 nucleotides that inhibit gene expression of RhoA and/or VEGF-C

It is an object of the present invention to provide siNA molecules that are site directed to a target gene.

It is an object of the present invention to provide siNA molecules that can be used for inhibiting angiogenesis.

It is an object of the present invention to provide short nucleic acid molecules, which can be used alone or in combination with other therapies, for effective management of ocular diseases or disorders such as diabetic retinopathy, age related macular degeneration (AMD), neo-vascular glaucoma, rubeosis, uveitis, choroidal neovascularization, eye infections such as conjunctivitis, keratitis, blepharitis, sty, chalazion, iritis, and stromal keratitis.

It is an object of the present invention to provide short nucleic acid molecules, which can be used alone or in combination with other therapies, for effective management of cancers such as breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, lymphoma, glioma, or multidrug resistant cancer.

It is an object of the present invention to provide short interfering nucleic acid molecules, which can be conjugated to compositions such as lipids, polymers and monoclonal antibodies.

### SUMMARY OF THE INVENTION

The present invention designs and presents nucleic acid molecules targeting RhoA and/or VEGF-C genes, which can specifically and effectively direct homology-specific post transcriptional gene silencing, and therefore are useful as highly effective, selective and potent therapeutics, with minimal side effects.

The present invention includes double stranded short nucleic acid (siNA) molecules that are specifically targeted. In some embodiments, the short nucleic acid molecules are RNA, including siRNA targeting RhoA and/or VEGF-C genes.

In one embodiment, the invention presents a siNA molecule that down-regulates expression of a VEGF-C gene, wherein the siNA molecule comprises about 19 to about 30 base pairs. In related embodiments the siNA is an siRNA that is 19, 20, 21, 22, 23, 24, 25, 26, 27, 28, 29 or 30 base pairs.

In one embodiment, the invention presents a siNA molecule that down-regulates expression of a RhoA gene, wherein the siNA molecule comprises about 19 to about 30 base pairs. In related embodiments the siNA is an siRNA that is 19, 20, 21, 22, 23, 24, 25, 26, 27, 28, 29 or 30 base pairs.

In another embodiment, the invention presents a siNA molecule comprising nucleotide sequence, for example, nucleotide sequence in the antisense region of the siNA molecule that is complementary to a nucleotide sequence or portion of sequence of a VEGF-C and/or RhoA gene. In another embodiment, the invention presents a siNA molecule comprising a region, for example, the antisense region of the siNA construct, complementary to a sequence comprising a VEGF-C and/or RhoA gene sequence or a portion thereof.

In one embodiment of the invention a siNA molecule comprises an antisense strand comprising a nucleotide sequence that is complementary to a nucleotide sequence or a portion thereof encoding a VEGF-C and/or a RhoA protein. In another embodiment, the siNA further comprises a sense strand, wherein said sense strand comprises a nucleotide sequence of a VEGF-C and/or a RhoA gene or a portion thereof.

In one embodiment, a siNA molecule of the invention has RNA interference (RNAi) activity that modulates expression of an RNA encoded by a VEGF-C and/or a RhoA gene.

In another embodiment, the invention presents a double-stranded short interfering nucleic acid (siNA) molecule that down-regulates expression of a VEGF-C and/or RhoA gene, wherein the siNA comprises an antisense region, complementary to a nucleotide sequence of the VEGF-C and/or RhoA gene or a portion thereof, and a sense region substantially similar to the nucleotide sequence of the VEGF-C and/or RhoA gene or a portion thereof. In one embodiment, the antisense region and the sense region each comprise about 19 to about 30 (e.g., 19, 20, 21, 22, 23, 24, 25, 26, 27, 28, 29, or 30) nucleotides, wherein the antisense region comprises at least 19 nucleotides that are complementary to nucleotides of the sense region.

In one embodiment, a siNA molecule of the invention comprises any of one of:

RINA 6 comprising sense strand SEQ ID NO: 7 and antisense strand SEQ ID NO: 8;

RINA 17 comprising sense strand SEQ ID NO: 9 and antisense strand SEQ ID NO: 10;

RINA 30 comprising sense strand SEQ ID NO: 11 and antisense strand SEQ ID NO: 12;

RINA 50 comprising sense strand SEQ ID NO: 13 and antisense strand SEQ ID NO: 14;

RINA 51 comprising sense strand SEQ ID NO: 15 and antisense strand SEQ ID NO: 16;

and RINA 52 comprising sense strand SEQ ID NO: 17 and antisense strand SEQ ID NO: 18.

In the present invention, the knockdown of VEGF-C expression using siNA leads to decreased activation of VEGFR-3, which is required for the initiation and sustaining of angiogenesis. Further, the present invention demonstrates for the first time that knockdown of VEGF-C also leads to decrease in expression of VEGF-A. Thus, the present invention establishes a direct relationship exists between expression levels of both VEGF-C and VEGF-A and thus provides a means to control or reverse angiogenesis.

The present disclosure provides short nucleic acid molecules for modulation of RhoA and/or VEGF-C gene expression. In related embodiments, the present invention provides RhoA and/or VEGF-C targeting short nucleic acid molecules for the inhibition of angiogenesis. Such molecules may be used alone (RhoA and/or VEGF-C targeting molecules), or in combination with other therapies, for the management and treatment of various disorders associated with excessive angiogenesis, such as age related macular degeneration and diabetic retinopathy, hypertension, cancer, inflammation and autoimmune diseases.

In some embodiments, the present invention provides siNAs having between 19 to 30 nucleotides, between 25 and 29 nucleotides, or having 27 nucleotides, where the sequence is designed for better stability and efficacy in knockdown (i.e., reduction) of RhoA and/or VEGF-C gene expression. Such siNAs can be used alone or in combination with other therapies.

In one embodiment, the nucleic acid molecule of the present invention comprises 19-30 nucleotides complementary to RNA corresponding to a RhoA and/or VEGF-C nucleic acid sequence. In related embodiments, the invention encompasses compounds, compositions and uses of 19, 20, 21, 22, 23, 24, 25, 26, 27, 28, 29 or 30-mers, including 27-mer siNA molecules, for modulation of RhoA and/or VEGF-C gene expression.

The present invention provides stable compositions of siNA with or without conjugation to cholesterol. The compounds of the present invention are useful in therapy of angiogenesis either alone or in combination with other treatments or therapies.

In certain embodiments, the siNA of the present invention includes short interfering RNA (siRNA), a double stranded RNA (dsRNA), a micro RNA (μRNA), and/or a short hairpin RNA (shRNA) molecule. The siNA molecules can be unmodified or modified chemically. In the some embodiments the present invention relates to a siRNA having 19, 20, 21, 22, 23, 24, 25, 26, 27, 28, 29 or 30 nucleotides on one strand.

Nucleotides of the present invention can be chemically synthesized, expressed from a vector, or enzymatically synthesized.

In one embodiment, an siRNA composition of the invention comprises combination of two double stranded siRNAs, wherein one strand of the first double stranded siRNA molecule is complimentary to a portion of an RNA encoding RhoA, and one strand of the second double stranded siRNA molecule is complimentary to a portion of an RNA encoding VEGF-C. In another embodiment, one siRNA molecule of the invention comprises a double stranded RNA, wherein one strand of the RNA comprises a portion of a sequence of RNA encoding RhoA, and another siRNA molecule comprises a double stranded RNA wherein one strand of the RNA comprises a portion of a sequence of RNA encoding VEGF-C.

In one embodiment, the invention targets RhoA and/or VEGF-C as set forth in GenBank Accession Numbers NM_{—}001664 and NM_{—}005429, respectively. The present invention is not limited, however, to nucleotides targeting one variant of RhoA, but also includes nucleotides that target RhoA-related molecules including single nucleotide polymorphisms of RhoA, RhoA homologs, and RhoA splice and transcript variants. The present invention also contemplates nucleotides that target genes involved in RhoA regulatory pathway as a means of regulating RhoA. Similarly, nucleotides targeting VEGF-C are also not limited to nucleotides targeting one variant of VEGF-C, but also include nucleotides that target VEGF-C-related molecules including single nucleotide polymorphisms of VEGF-C, VEGF-C homologs, and VEGF-C splice and transcript variants. The present invention also contemplates nucleotides that target genes involved in VEGF-C regulatory pathway as a means of regulating VEGF-C.

In other embodiments, the present invention provides compositions and methods used to regulate RhoA and/or VEGF-C expression or activity. RhoA expression or activity may be regulated by a small nucleic acid molecule that targets RhoA directly, or by targeting molecules that regulate the RhoA pathway. VEGF-C expression or activity may be regulated by a small nucleic acid molecule that targets VEGF-C directly, or by targeting molecules which regulate the VEGF-C pathway. Small nucleic acid molecules that target RhoA and/or VEGF-C may be used in combination with other small nucleic acid molecules or small chemical molecules.

In related embodiments, the targeting of RhoA and/or VEGF-C is used to regulate neovascular disease states that respond to modulation of RhoA and/or VEGF-C expression levels in the cell, such as age related macular degeneration, diabetic retinopathy and glaucoma, cancer, inflammation and autoimmune diseases.

In some embodiments, chemically synthesized siNAs of 27 nucleotides in length are used to reduce expression levels of RhoA and VEGF-C, alone or in combination with other small nucleic acid molecules directed against genes that are involved in treatment of the same disease. In other embodiments the siNAs are of 19, 20, 21, 22, 23, 24, 25, 26, 28, 29 or 30 nucleotides.

In another embodiment, the present invention provides techniques for validating the efficacy of siNA, using biomarkers for angiogenesis, such as expression levels of VEGF-A, and phosphorylation status of ROCK-1 and ROCK-2.

In one embodiment, the invention presents a mammalian cell, for example a human cell, comprising a small nucleic acid molecule of the invention.

The present invention presents a method of down-regulating (also called “knocking down”) RhoA and/or VEGF-C activity in a cell, comprising contacting the cell with a nucleic acid molecule of the invention, under conditions suitable for down-regulating RhoA and VEGF-C activity.

In one embodiment, the present invention also presents a method for treating a subject having a condition associated with elevated levels of RhoA and/or VEGF-C, comprising contacting cells of the subject with a nucleic acid molecule of the invention, under conditions suitable for the treatment. In related embodiments, the method is supplemented with a drug therapy for the same condition.

In further related embodiments, the present invention also presents a method using a therapeutic agent for the treatment of ocular diseases or disorders such as anterior and posterior ocular diseases.

In one embodiment, the present invention provides a combination of short nucleic acid molecules which inhibit the expression of at least one gene associated with neovascularization and angiogenesis. The present invention provides short nucleic acid molecules for treatment of diabetic retinopathy, age related macular degeneration (AMD), wet AMD, inflammatory conditions of the eye such as uveitis, rubeosis, conjunctivits, keratitis, blepharitis, sty, chalazion, iritis, stromal keratitis, and cancers.

In another embodiment, the present invention provides short nucleic acid molecules for the treatment of cancer, such as breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, lymphoma, glioma, or multidrug resistant cancer, comprising administering to a subject a nucleic acid molecule of the invention under conditions suitable for said treatment.

In another embodiment, the invention provides a method to prevent activation of VEGFR-3 nullified by inhibiting its interacting ligand, VEGF-C.

In one embodiment, the present invention establishes that a direct correlation exists between expression levels of VEGF-C and VEGF-A. Likewise, the present invention establishes that a homeostasis exists between expression levels of VEGF-C and VEGF-A, which is required for the initiation and containment of neoangiogenesis.

In another embodiment, other drug therapies contemplated by the invention include chemical inhibitiors, monoclonal antibodies or a combination thereof.

The present invention presents compositions comprising the nucleic acid molecules of the invention in a pharmaceutically acceptable carrier.

The invention also presents a method of administering to a cell, such as mammalian cell (e.g., a human cell), a nucleic acid of the invention. Such a cell can be in culture or in a mammal, such as a human. The method of administering comprises contacting the cell with a nucleic acid molecule of the invention under conditions suitable for such administration. The method of administration may be in the presence of a delivery reagent, for example a lipid, cationic lipid, phospholipid, or liposome. The site of administration may be selected depending on the disease, e.g., ocular diseases, and the method of delivery may be, for example, subconjunctival, intravenous, subcutaneous, eye drops and/or topical.

In one embodiment, the present invention provides compositions comprising: (1) a first short nucleic acid molecule that modulates VEGF-C expression, wherein the first short nucleic acid molecule comprises a first nucleotide sequence, wherein a sequence of at least 19 contiguous nucleotides in the first nucleotide sequence is at least 95% complementary to a sequence selected from the group consisting of SEQ ID NO: 1, SEQ ID NO: 2 and SEQ ID NO: 3, and/or (2) a second short nucleic acid molecule that modulates RhoA expression, wherein the second short nucleic acid molecule comprises a second nucleotide sequence, wherein a sequence of at least 19 contiguous nucleotides in the second nucleotide sequence is at least 95% complementary to a sequence selected from the group consisting of SEQ ID NO: 4, SEQ ID NO: 5 and SEQ ID NO: 6. See Table 1 below.

In one embodiment, a sequence of at least 19 contiguous nucleotides in the first nucleotide sequence is completely (100%) complementary to a sequence selected from the group consisting of SEQ ID NO: 1, SEQ ID NO: 2 and SEQ ID NO: 3, and a sequence of at least 19 contiguous nucleotides in the second nucleotide sequence is completely (100%) complementary to a sequence selected from the group consisting of SEQ ID NO: 4, SEQ ID NO: 5 and SEQ ID NO: 6.

In another embodiment, a first short nucleic acid molecule comprises a siNA selected from the group consisting of: RINA 6 comprising sense strand SEQ ID NO: 7 and antisense strand SEQ ID NO: 8; RINA 17 comprising sense strand SEQ ID NO: 9 and antisense strand SEQ ID NO: 10; and RINA 30 comprising sense strand SEQ ID NO: 11 and antisense strand SEQ ID NO: 12. See Table 2 below.

In another embodiment, a second short nucleic acid molecule comprises a siNA selected from the group consisting of: RINA 50 comprising sense strand SEQ ID NO: 13 and antisense strand SEQ ID NO: 14; RINA 51 comprising sense strand SEQ ID NO: 15 and antisense strand SEQ ID NO: 16; and RINA 52 comprising sense strand SEQ ID NO: 17 and antisense strand SEQ ID NO: 18. See Table 2 below.

In another embodiment, a first short nucleic acid molecule comprises RINA 30 comprising sense strand SEQ ID NO: 11 and antisense strand SEQ ID NO: 12, and the second short nucleic acid molecule comprises RINA 52 comprising sense strand SEQ ID NO: 17 and antisense strand SEQ ID NO: 18.

In certain embodiments, the present invention also provides compositions for the treatment of an ocular disorder comprising one of the compositions described herein, and a pharmaceutically acceptable carrier. In one embodiment, the ocular disorder is retinopathy or glaucoma. In other embodiments, the present invention relates to compositions for the treatment of a neovascular disease comprising one of the compositions described herein, and a pharmaceutically acceptable carrier.

In other embodiments, compositions of the invention comprise a lipid, polymer and/or a monoclonal antibody. In another embodiment, at least one short nucleic acid molecule is conjugated to cholesterol.

In other embodiments, the invention comprises methods of inhibiting VEGF-C, VEGF-A, and/or RhoA by administering a siRNA, including the siRNA's of the invention, and related methods of use and methods of treatment. In one embodiment, the method comprises comprising contacting the cell with a short nucleic acid molecule comprising a sequence of at least 19 contiguous nucleotides that is at least 95% complementary to a sequence of at least 19 contiguous nucleotides within a full length VEGF-C or RhoA gene. In other embodiments, the sequence is 100% complementary. In other embodiments, the nucleic acid is 19, 20, 21, 22, 23, 24, 25, 26, 27, 28, 29 or 30 nucleotides on one strand. In one embodiment, the short nucleic acid molecule comprises a antisense strand consisting of 27 nucleotides, wherein the 27 nucleotide strand sequence is at least 95% complementary to 27 contiguous nucleotides within a full length VEGF-C or RhoA gene.

In other embodiments, the present invention provides methods for reducing or down-regulating VEGF-A expression, or VEGF-A secretion from a cell, comprising contacting the cell with a short nucleic acid molecule comprising a sequence of at least 19 contiguous nucleotides that is at least 95% complementary to a sequence of at least 19 contiguous nucleotides within a full length VEGF-C or RhoA gene. In other embodiments, the sequence is 100% complementary. In other embodiments, the nucleic acid is 19, 20, 21, 22, 23, 24, 25, 26, 27, 28, 29 or 30 nucleotides on one strand. In one embodiment, the short nucleic acid molecule comprises a antisense strand consisting of 27 nucleotides, wherein the 27 nucleotide strand sequence is at least 95% complementary to 27 contiguous nucleotides within a full length VEGF-C or RhoA gene.

In certain embodiments, upon performing methods such as those described herein, VEGF-A secretion is reduced or down-regulated by about 50% in a cell after contacting the cell with a short nucleic acid molecule at a concentration of about 0.6 nM.

In certain embodiments the short nucleic acid molecule (1) directly modulates expression of VEGF-C or RhoA; (2) inhibits VEGF-C and/or RhoA expression; (3) inhibits VEGF-A expression or secretion; and/or (4) binds to at least one molecule that modulates expression or secretion of VEGF-A.

In certain embodiments of the herein mentioned methods, the first short nucleic acid molecule comprises a sequence of at least 19 contiguous nucleotides that is at least 95% to a sequence of at least 19 contiguous nucleotides within a full length VEGF-C gene, and the second short nucleic acid molecule comprises a sequence of at least 19 contiguous nucleotides that is at least 95% to a sequence of at least 19 contiguous nucleotides within a full length RhoA gene. In related embodiments, the nucleic acid is 19, 20, 21, 22, 23, 24, 25, 26, 27, 28, 29 or 30 nucleotides in length. In other related embodiments, the sequence is completely (100%) complementary.

In certain embodiments of the herein mentioned methods, the siRNA molecules are selected from:

RINA 6 comprising sense strand SEQ ID NO: 7 and antisense strand SEQ ID NO: 8;

RINA 17 comprising sense strand SEQ ID NO: 9 and antisense strand SEQ ID NO: 10;

RINA 30 comprising sense strand SEQ ID NO: 11 and antisense strand SEQ ID NO: 12;

RINA 50 comprising sense strand SEQ ID NO: 13 and antisense strand SEQ ID NO: 14;

RINA 51 comprising sense strand SEQ ID NO: 15 and antisense strand SEQ ID NO: 16;

and RINA 52 comprising sense strand SEQ ID NO: 17 and antisense strand SEQ ID NO: 18.

In other embodiments, the present invention provides methods for reducing or down-regulating VEGF-A secretion from a cell comprising contacting the cell with a short nucleic acid molecule comprising a sequence of at least 19 contiguous nucleotides that is completely (100%) complementary to a sequence of at least 19 contiguous nucleotides within a full length VEGF-C or RhoA gene. In related embodiments, the nucleic acid is 19, 20, 21, 22, 23, 24, 25, 26, 27, 28, 29 or 30 nucleotides in length. In one embodiment, the short nucleic acid molecule comprises a antisense strand consisting of 27 nucleotides, wherein the 27 nucleotide strand sequence is completely (100%) complementary to 27 contiguous nucleotides within a full length VEGF-C or RhoA gene.

In other embodiments, the present invention provides methods for reducing or down-regulating VEGF-A secretion from a cell comprising contacting the cell with a short nucleic acid molecule comprising a nucleotide sequence that is at least 95% complementary to a sequence selected from the group consisting of SEQ ID NO: 1, SEQ ID NO: 2 and SEQ ID NO: 3. In one embodiment, the short nucleic acid molecule comprises a nucleotide sequence that is completely (100%) complementary to a sequence selected from the group consisting of SEQ ID NO 1, SEQ ID NO: 2 and SEQ ID NO: 3.

In other embodiments, the present invention provides methods for reducing or down-regulating VEGF-A secretion from a cell comprising contacting the cell with a short nucleic acid molecule comprising a nucleotide sequence that is at least 95% complementary to a sequence selected from the group consisting of SEQ ID NO: 4, SEQ ID NO: 5 and SEQ ID NO: 6. In one embodiment, the short nucleic acid molecule comprises a nucleotide sequence that is completely (100%) complementary to a sequence selected from the group consisting of SEQ ID NO: 4, SEQ ID NO: 5 and SEQ ID NO: 6.

In other embodiments, the present invention provides methods for reducing or down-regulating VEGF-A secretion from a cell comprising contacting the cell with a first short nucleic acid molecule and second short nucleic acid molecule, wherein the first short nucleic acid molecule comprises a sequence of at least 19 contiguous nucleotides that is at least 95% complementary to a sequence of at least 19 contiguous nucleotides within a full length VEGF-C gene, and wherein the second short nucleic acid molecule comprises a sequence of at least 19 contiguous nucleotides that is at least 95% complementary to at least 19 contiguous nucleotides within a full length RhoA gene.

In certain embodiments of the herein mentioned methods, the first short nucleic acid molecule comprises a sequence of at least 19 contiguous nucleotides that is completely (100%) complementary to a sequence of at least 19 contiguous nucleotides within a full length VEGF-C gene, and the second short nucleic acid molecule comprises a sequence of at least 19 contiguous nucleotides that is completely (100%) complementary to a sequence of at least 19 contiguous nucleotides within a full length RhoA gene.

In certain embodiments, the present invention also provides methods for treating an ocular disorder, neovascular disease, and/or cancer comprising administering at least one of the compositions described herein.

In other embodiments, the present invention provides methods for reducing or inhibiting angiogenesis or neoangiogenesis in a tissue comprising contacting the tissue with a first short nucleic acid molecule and second short nucleic acid molecule, wherein the first short nucleic acid molecule comprises a sequence of at least 19 contiguous nucleotides that is at least 95% complementary to at least 19 contiguous nucleotides within a full length VEGF-C gene, and wherein the second short nucleic acid molecule comprises a sequence of at least 19 contiguous nucleotides that is at least 95% complementary to a sequence of at least 19 contiguous nucleotides within a full length RhoA gene. In certain embodiments, the first short nucleic acid molecule comprises a sequence of at least 19 contiguous nucleotides that is completely (100%) complementary to a sequence of at least 19 contiguous nucleotides within a full length VEGF-C gene, and the second short nucleic acid molecule comprises a sequence of at least 19 contiguous nucleotides that is completely (100%) complementary to a sequence of at least 19 contiguous nucleotides within a full length RhoA gene. In related embodiments, the nucleic acid is 19, 20, 21, 22, 23, 24, 25, 26, 27, 28, 29 or 30 nucleotides in length

In other embodiments, the present invention presents methods for reducing or inhibiting angiogenesis or neoangiogenesis in a tissue comprising contacting the tissue with at least one of the compositions described herein.

In other embodiments, the present invention provides methods for reducing or inhibiting ROCK phosphorylation, such as ROCK-2 phosphorylation, in a cell comprising contacting the cell with a short nucleic acid molecule comprising a sequence of at least 19 contiguous nucleotides that is at least 95% complementary to a sequence of at least 19 contiguous nucleotides within a full length RhoA gene, wherein phosphorylation of ROCK-2 is reduced or inhibited, but ROCK expression is not knocked down. In one related embodiment, the short nucleic acid molecule modulates RhoA expression and comprises a nucleotide sequence of at least 19 contiguous nucleotides at least 95% complementary to a sequence selected from the group consisting of SEQ ID NO: 4, SEQ ID NO: 5 and SEQ ID NO: 6. In another related embodiment, the short nucleic acid molecule comprises a siNA selected from the group consisting of: RINA 50 comprising sense, strand SEQ ID NO: 13 and antisense strand SEQ ID NO: 14; RINA 51 comprising sense strand SEQ ID NO: 15 and antisense strand SEQ ID NO: 16; and RINA 52 comprising sense strand SEQ ID NO: 17 and antisense strand SEQ ID NO: 18.

### BRIEF DESCRIPTION OF THE DRAWINGS

The following drawings form part of the present specification and are included to further demonstrate certain aspects of the present disclosure.

FIG. 1: Reverse transcriptase PCR products amplified by gene specific primers were resolved over 2% agarose gel electrophoresis and stained with ethidium bromide to visualize the amplicons of interest. Arrowheads indicate amplicons of specific gene products, showing that two cell lines, HUVEC (Human Umblical Vascular Endothelial Cells, ATCC) and PC3 (Prostate Cancer cells, ATCC), express both VEGF-C and RhoA.

Lane 1: PC3 VEGF-C;

Lane 2: PC3 VEGF-C no template (c-DNA) added;

Lane 3: PC3 RhoA;

Lane 4: PC3 RhoA no template (c-DNA) added;

Lane 5: 100 bp ladder;

Lane 6: HUVEC VEGF-C;

Lane 7: HUVEC VEGF-C no template (c-DNA) added:

Lane 8: HUVEC RhoA;

Lane 9: HUVEC RhoA no template (c-DNA) added;

Lane 10: 100 bp ladder.

FIG. 2: Standard curve for VEGF-A concentration determination, as obtained from a sandwich ELISA kit and using standards for VEGF-A provided in the kit.

FIG. 3: FIG. 3A. Knockdown of VEGF-C at various concentrations of RINA 30 ranging from 0.01 to 100 nM, at 10-fold increments, where results reach a plateau at 70% inhibition. The IC50 value is 0.5 nM for RINA 30. FIG. 3B. Knockdown of VEGF-A at various concentrations of RINA 30 ranging from 0.01 to 100 nM, at 10-fold increments, where results reach a plateau at 48% inhibition. The IC50 value is 0.8 nM for RINA 30.

FIG. 4: FIG. 4A. Retinal pigmented cells were transfected with RhoA RINAs 50, 51 and 52. Protein from lysed transfected cells were blotted onto pre-wet nitrocellulose membrane. Phosphorylated ROCK-2 was detected on the nitrocellulose using an anti-phospho-Rho-associated kinase-alpha antibody ABCAM ab 24843 (ABCAM Plc, Cambridge Mass., USA) and an ALP-conjugated secondary antibody, anti-mouse gamma chain specific ALP conjugate (Sigma). Arrowheads indicate the 160 KDa phosphorylated ROCK-2 protein band, and the 51 KDa tubulin protein band as an internal control, used to ensure that equal quantities of protein were loaded in all wells.

Lane 1: RINA 50 transfected cells,

Lane 2: RINA 51 transfected cells;

Lane 3: RINA 52 transfected cells;

Lane 4: Negative RINA tranfected cells;

Lane 5: Untreated RPE-19 cells;

M represents molecular weight markers.

FIG. 4B. Retinal pigmented cells were transfected with RINA 52 and protein from lysed transfected cells was blotted onto pre-wet nitrocellulose membrane. ROCK-1 was detected on the nitrocellulose using an anti-ROCK-1 mouse monoclonal IgG1 (Santa Cruz) and ALP-conjugated secondary antibody, anti-mouse gamma chain specific ALP conjugate (Sigma). Arrowheads indicate the 160 KDa ROCK-1 protein band, and the 51 KDa tubulin protein band (internal control, used to ensure that equal quantities of protein were loaded in all wells). Protein levels of ROCK-1 remained quantitatively similar, indicating that knockdown by RINA 52 inhibited phosphorylation of ROCK-2, but not quantitative levels of ROCK-1.

Lane 1: Untreated cells;

Lane 2: Negative RINA transfected cells;

Lane 3: RINA 52 transfected cells.

FIG. 5: Untreated HUVEC cells, negative RINA treated and RINA 30 treated HUVEC cells were allowed to form vessels on extracellular matrix (ECM) coated wells of a 96-well plate. After 8 h of incubation on ECM, light microscopic observations were made and photographs were taken at 10× magnification. FIG. 5A shows untreated HUVEC cells initiating pentagonal network of vessels. Arrowhead indicates the branching of vessels. FIG. 5B shows negative RINA treated cells forming vessels. Arrowhead indicates vessels formed. FIG. 5C shows RINA 30 treated cells. Cells separated from each other, and exhibited no signs of cell migration or initiation of vessel formation.

FIG. 6. VEGF after knockdown. Decreased VEGF-A production with the increase in concentration of siRNA462 in RPE19 (FIG. 6A) as well as MCF7 cell lines (FIG. 6B) However, siRNA462 in both cell lines caused an increase in secreted VEGF-C with concentration. FIG. 6C shows MCF7.

FIG. 7A-D. RPE19 cells were knocked down with siRNA462, RINA 30 or both under hyperglycemic condition or normal conditions and examined for their affect on VEGF-A and VEGF-C secretion.

FIG. 7A. Effect of siRNAs on VEGF-A secretion under hyperglycemia or normoglycemia.

FIG. 7B. Effect of siRNAs on VEGF-C secretion under hyperglycemia or normoglycemia.

FIG. 7C. Effect of siRNAs on VEGF-A secretion under hypoxia or normaxia.

FIG. 7D. Effect of siRNAs on VEGF-C secretion under hypoxia or normaxia.

FIG. 8. RPE19 and MCF7 cells were transfected with RINA 30 at various concentrations (ranging from 0.01 -100 nM with one log increment each) and analyzed cell culture supernatants at the end of 72 h post transfection. Analysis of cell culture supernatants for VEGF-C ELISA have shown decrease in secretary levels of VEGF-C with the increase in concentration of RINA30 in RPE19 (FIG. 8A) as well as MCF7 (FIG. 8B) cells. Similarly the secretary levels of VEGF-A were also found to be decreased with the increase in concentration of RINA30 in both cell lines. FIG. 8C shows RPE19; and FIG. 8D shows MCF7.

FIG. 9. HUVEC were transfected with RINA52 and analyzed for their ability to reduce protein levels and its effect on angiogenesis. FIG. 9A shows protein blot analysis of HUVEC cells for RhoA protein knockdown after transfection with RINA52 (0.01-100 nM concentrations with one log increment each) at the end of 72 h post transfection. Figure FIG. 9B-C shows proliferation after knockdown by RINA52, with or without supplementation with external. VEGF-A or VEGF-C. UT=untreated with siRNA. FIG. 9B shows HUVEC cells, and FIG. 9C shows HMVEC cells.

FIG. 9D shows the % inhibition of angiogenesis by HUVEC cells associated with treatment of cells with siRNA (RINA52), and that this affect cannot be reversed by external supplementation with VEGF-A and/or VEGF-C.

FIG. 10. HUVEC cells transfected. FIG. 10A shows cells knocked down for RhoA where the arrowhead indicates absence of F-actin filaments. FIG. 10B shows cells transfected with negative RINA where arrowhead indicates intact F-actin filaments.

FIG. 10C shows untransfected cells where F-actin filaments were prominent.

FIG. 11. Fold change in secreted VEGF-A (FIG. 11A) and VEGF-C (FIG. 11B) at different glucose concentrations in cell culture media.

FIG. 12. HUVEC cell proliferation in response to addition of VEGF-A or VEGF-C to culture supernatant. VEGF-C (A), VEGF-A (s).

FIG. 13. Knockdown of VEGF-C or VEGF-A expression following transfection with different siRNA targeting VEGF-C.

### DETAILED DESCRIPTION OF THE INVENTION

### Definitions

The term “short nucleic acid molecule” refers to any nucleic acid molecule capable of modulating gene expression. The terms “short interfering nucleic acid”, “siNA” or “siNA molecules,” “short interfering nucleic acid molecule,” or “short interfering oligonucleotide molecule” refer to any nucleic acid molecule capable of inhibiting, down-regulating or knocking down gene expression.

Typically, short interfering nucleic acid molecules are composed primarily of RNA, and may be referred to as “short interfering RNA” or “siRNA.” A siNA may, however, include nucleotides other than RNA, such as in DNAi (interfering DNA), or other modified bases. Thus, the term “RNA” as used herein means a molecule comprising at least one ribonucleotide residue and includes double stranded RNA, single stranded RNA, isolated RNA, partially purified, pure or synthetic RNA, recombinantly produced RNA, as well as altered RNA such as analogs or analogs of naturally occurring RNA.

The term “RINA” refers to siRNA duplexes, having sense and antisense strands. RINA 6, RINA 17, RINA 30, RINA 50, RINA 51 and RINA 52 refers to specific siRNA duplexes having certain nucleotide sequences, as presented in Table 2 below.

The term “negative RINA” is a commercially available negative control comprises of a scrambled sequence, obtained. from Ambion Inc.

The term “modulate” or “modulates” means that gene expression or level of RNA molecule or equivalent RNA molecules encoding one or more protein or protein subunits or peptides, or activity of one or more protein subunits or peptides, is up-regulated or down-regulated such that the expression, level, or activity is greater than or less than that observed in the absence of the modulator. The term “modulate” includes “inhibit.”

The terms “inhibition” (or “inhibit”), “down-regulation” (or “down-regulate”) or “knockdown” of a gene means that expression of a gene, or level of RNA molecules or equivalent RNA molecules encoding one or more proteins or protein subunits, or activity of one or more proteins or protein subunits, is reduced below that observed in the absence of an inhibitory nucleic acid molecule (e.g., siNA) of the present invention. In one embodiment, inhibition or down-regulation observed in the presence of one or more siNA molecules is greater than inhibition or down-regulation observed in the absence of the siNA(s) or in the presence of, for example, a siNA molecule, with a scrambled sequence or with mismatches. Likewise, in one embodiment, expression of a gene or protein is “knocked down” in the presence of one or more siNA molecules, as compared to gene or protein expression observed in absence of the siNA molecule(s), or in the presence of, for example, a siNA molecule with a scrambled sequence or with mismatches.

The term “gene” refers to a nucleic acid that encodes an RNA, for example, nucleic acid sequences including, but not limited to, a structural gene encoding a polypeptide.

The term “RhoA” as used herein refers to any RhoA protein, peptide, or polypeptide or a derivative thereof, such as encoded by Genbank Accession number NM_{—}001664, having RhoA activity. The term RhoA also refers to nucleic acid sequences encoding any RhoA protein, peptide, polypeptide, or polypeptide having isoforms, mutant genes, splice variants or polymorphisms, having RhoA activity.

The term “ROCK” as used herein refers to any Rho kinase (e.g., ROCK-1, ROCK-2) protein, peptide, or polypeptide or a derivative thereof, such as encoded by Genbank Accession numbers NM_{—}005406 (ROCK-1) and NM_{—}004850 (ROCK-2) having Rho kinase activity. The term ROCK also refers to nucleic acid sequences encoding any RhoA kinase protein, peptide, polypeptide, or polypeptide having isoforms, mutant genes, splice variants or polymorphisms, having RhoA kinase activity.

The term “VEGF” as used herein refers to any vascular endothelial growth factor (e.g., VEGF, VEGF-A, VEGF-B, VEGF-C, VEGF-D) protein, peptide, or polypeptide having vascular endothelial growth factor activity. The term VEGF also refers to nucleic acid sequences encoding any vascular endothelial growth factor protein, peptide, polypeptide, or polypeptide having isoforms, mutant genes, splice variants or polymorphisms, having vascular endothelial growth factor activity.

The term “VEGF-C” as used herein refers to any protein, peptide, or polypeptide or a derivative thereof, such as encoded by Genbank Accession number NM_{—}005429, having vascular endothelial growth factor type C activity. The term VEGF-C also refers to nucleic acid sequences encoding any VEGF-C protein, peptide, polypeptide, or polypeptide having isoforms, mutant genes, splice variants or polymorphisms, having VEGF-C activity.

The term “VEGF-A” as used herein refers to any protein, peptide, or polypeptide or a derivative thereof, such as encoded by Genbank Accession number NM_{—}001025366, having vascular endothelial growth factor type A activity. The term VEGF-A also refers to nucleic acid sequences encoding any VEGF-A protein, peptide, polypeptide, or polypeptide having isoforms, mutant genes, splice variants or polymorphisms, having VEGF-A activity.

The terms “target gene” or “target nucleic acid” as used herein means any nucleic acid sequence whose expression or activity is to be modulated. The target nucleic acid can be DNA or RNA.

The term “sense region” as used herein with regard to a siNA of the present invention refers to a nucleotide sequence of a siNA molecule that is complementary to an antisense region of the siNA molecule. In addition, the sense region of a small nucleic kid molecule can comprise a nucleic acid sequence having homology with a target nucleic acid sequence.

The term “antisense region” as used herein with regard to a siNA of the present invention means a nucleotide sequence of a siNA molecule that is complementary to a target nucleic acid sequence. It can also comprise a nucleic acid sequence that is complementary to a sense region of a siNA molecule.

The term “complementarily” or “complementary” as used herein means that a nucleic acid sequence can form hydrogen bond(s) with another nucleic acid sequence. A nucleic acid molecule comprising two or more nucleic acids may be partially or completely (100%) complementary to another nucleic acid molecule, for example, with regard to corresponding nucleic acids that are capable of forming a double stranded molecule. A percent “complementarity” or “complementary” indicates the percentage of contiguous residues in a nucleic acid molecule that can form hydrogen bonds with a second nucleic acid sequence. For instance, a first sequence is 95% complementary to a second sequence if 19 out of 20 contiguous nucleotides in the first sequence form hydrogen bonds with 19 out of 20 contiguous nucleotides in the second sequence. “Completely complementary” means that all contiguous residues of a nucleic acid sequence will hydrogen bond with the same number of contiguous residues in a second nucleic acid sequence. For example, a 23-mer nucleic acid may be completely (100%) complementary to a 27-mer nucleic acid with regard to 23 contiguous nucleic acids.

The term “ocular disorder” as used herein refers to any disease or disorder that affects the eye, such as anterior and posterior ocular diseases, including retinopathy, age related macular degeneration (AMD), diabetic retinopathy, glaucoma, inflammatory conditions of the eye, such as uveitis, rubeosis, conjunctivits, keratitis, blepharitis, sty, chalazion, iritis, stromal keratitis, and cancers.

The term “neovascular disease” as used herein refers to any disease or disorder involving abnormal neovascularization or neoangiogenesis, including cancers and ocular neovascular diseases such as retinal neovascularization and choroidal neovascularization.

The term “retinopathy” as used herein refers to any type of retinopathy such as angiopathic retinopathy, arteriosclerosis retinopathy, central angiospastic retinopathy, central serous retinopathy, circinate retinopathy, diabetic retinopathy, dysproteinemic retinopathy, hypertensive retinopathy, leukemic retinopathy, lipemic retinopathy, proliferative retinopathy, renal retinopathy, sickle cell disease, retinopathy, toxemic retinopathy of pregnancy and the like.

The term “glaucoma” as used herein includes primary open angle glaucoma, normal pressure glaucoma, hypersecretion glaucoma, ocular hypertension, acute angle closure glaucoma, chronic angle closer glaucoma, plateau iris syndrome, combined-mechanism glaucoma, steroid glaucoma, capsular glaucoma, pigmentary glaucoma, secondary glaucoma associated with amyloidosis, neovascular glaucoma, malignant glaucoma and the like. Methods and compositions of the present invention for prophylaxis and treatment of glaucoma may have intraocular pressure lowering action, optic disc blood flow improving action and/or aqueous humor outflow promoting action.

The term “angiogenesis” refers to the generation of new blood supply, e.g., blood capillaries, vessels, and veins, e.g., from existing blood vessel tissue (e.g., vasculature). The process of angiogenesis can involve a number of tissue cell types including, for example, endothelial cells that form a single cell layer. The term “neoangiogenesis” refers to the proliferation of new blood capillaries, vessels, and veins in tissue. It also refers to the abnormal or excessive formation of new blood capillaries, vessels, and veins in tissue. Neoangiogenesis differs from angiogenesis in that angiogenesis usually involves the protrusion and outgrowth of capillary buds and sprouts from pre-existing blood vessels.

The term “cancer” or “proliferative disease” as used herein means any disease, condition, trait, genotype or phenotype characterized by unregulated cell growth or replication as is known in the art. It can include all types of cancer, tumors, lymphomas, carcinomas that can respond to the modulation of disease-related VEGF-C and/or RhoA gene expression in a cell or tissue alone or in combination with other therapies. In one embodiment, cancers associated with the eye are contemplated.

Previous work has examined the inhibition of Rho kinases, such as ROCK-1 and ROCK-2. Other separate work has examined the inhibition of VEGF-C. The present invention discloses the combination/synergistic approach of inhibiting both Rho kinases (via RhoA) and VEGF-C via small nucleic acids. In one embodiment, the present invention knocks down VEGF-C gene expression, by which one can reduce VEGF-C protein levels and thus reverse anti-apoptosis mechanisms activated by this factor. Further, the invention establishes for the first time that knockdown (i.e., reduction) of VEGF-C expression decreases levels of VEGF-A, which is primarily required for angiogenesis. The present invention establishes that both VEGF-C and VEGF-A secretions are interdependent and remain in direct proportion to each other quantitatively. Because the knockdown of VEGF-C inhibits the secretion of VEGF-A, such knockdown help arrest angiogenesis.

Further, the present invention establishes that RhoA plays an important role in regulation of VEGF-induced endothelial cell migration by phosphorylating the RhoA effector molecules ROCK-1 and ROCK-2. The present invention knocks down RhoA, thereby inhibiting (i.e., reducing) the signaling mechanisms of RhoA, which are responsible for endothelial cell migration and cell adhesion. In one embodiment, the present invention is directed to short nucleic acids targeting RhoA and/or VEGF-C genes that can specifically and effectively direct homology-specific post transcript gene silencing, and therefore act as highly effective, selective and potent therapeutics, with minimal side effects.

The present invention provides compounds, compositions and methods for the treatment of angiogenesis. The invention uses siNA-mediated inhibition of RhoA and VEGF-C.

In one embodiment, the present invention provides a combination of nucleic acids targeting RhoA gene and/or VEFG-C gene.

In one embodiment, the present invention targets RhoA and therefore inhibits the activation of its effector molecules, e.g., ROCK-1 and ROCK-2. Likewise, in one embodiment, the present invention knocks down RhoA protein levels and thus limits its ability to phosphorylate ROCK-1 and ROCK-2, but does not inhibit protein expression of ROCK-1 and ROCK-2.

In another embodiment, the present invention down regulates the protein levels of RhoA and thus inhibits VEGF-driven endothelial cell migration. In another embodiment, the present invention knocks down RhoA in the trabecular meshwork, and thus inhibits the intraocular pressure by regulating stress fiber formation.

In another embodiment, the present invention knocks down VEGF-C expression and secretion, which causes a reduction in the secretion of VEGF-A from cells.

### siNA Design And Testing

In one example, the present invention provides short interfering nucleic acid (siNA) molecules and their uses in modulation of RhoA and VEGF-C gene expression. The main features of the design studies are as follows:

1. Design of siNA;

2. Preparation of siNA;

3. Efficacy testing of the compounds;

4. Comparative data of siRNA having 21, 22, and 27 nucleotides, as examples of 19-30 nucleotides; and

5. Potency evaluation in animal models.

In one example, design of suitable siNA involved the design of the siRNA with 21, 23, and 27 nucleotides for modulation of RhoA and VEGF-C, without chemical modification. Target genes, such as RhoA and VEGF-C, were screened for accessible sites and siRNA were synthesized considering the open reading frame (ORF) sequences of RhoA and VEGF-C.

The following general requirements were considered in siNA design:

i. No runs of four or more A, T, G, or U in a row

ii. The following sequences were avoided, as they can induce an interferon response. A) 5′-UGUGU-3′ and B) 5′-GUCCUUCAA-3′

iii. The first 200 bases were omitted from the start codon to avoid binding to regulatory element.

iv. Each siNA duplex was checked in silico to avoid silencing of off-target effects made on BLAST search considering the following parameters:

A. Low complexity filtering was removed to avoid insignificance by BLAST resulting in limited or no query sequencer;

B. Word size was set to 7 letters, the minimal value algorithm;

C. Expected value threshold was set at 1000 to avoid the probability of short sequence occurrence.

Synthesis of siNA was done by commercially available methods (e.g., Qiagen) using chemically-protected phosphoramidite monomers. Resultant oligomers were purified by PAGE, desalting, or IE-HPLC. The quality of each siNA was analyzed by MALDI-TOF and yields were determined by an integrated spectrophotometer.

### Direct Assays On Cells

The ability of siRNAs to reduce the secretary levels of VEGF-C and VEGF-A were determined by sandwich ELISA in various cell lines including apical retinal pigmented epithelial cells.

IC50 values for RINA 30 transfected cells may be determined by estimating quantities of VEGF-C and VEGF-A using sandwich ELISA in breast cancer cell lines VEGF-C amounts are calculated on the basis of standard graph for VEGF-C (graph not provided). Percentage VEGF-C knockdown was calculated as the proportion of VEGF-C secreted in siRNA treated sample against control.

The cytotoxicity of siRNAs on cells may be analyzed by measuring amount of LDH released into the medium due to cell necrosis. The effect of siRNAs on various cytokines being released from the cells may be estimated using a cytokine array kit.

The angiogenic or vessel formation ability of endothelial cells were analyzed upon knocking down of VEGF-C and RhoA with the corresponding siRNA under in-vitro conditions either alone or in combination. In particular, the ability of VEGF-A alone to induce vessel formation, after VEGF-C and RhoA knockdown, was analyzed using an in vitro angiogenesis assay using Human Umbilical Vein Endothelial Cells (HUVEC) cells (ATCC). The ability of VEGF-C alone to induce angiogenesis in absence of VEGF-A and RhoA was also analyzed under in vitro conditions employing HUVEC cells. For example, RhoA expression was knocked down using siNAs and VEGF-A was not added. In addition, RhoA expression was knocked down using siNAs and VEGF-A is neutralized using VEGF-A receptors The ability of VEGF-C and VEGF-A together to induce angiogenesis in the absence of RhoA can be analyzed under in vitro conditions.

Further, the effect of RhoA knockdown using siNA was determined by observing the formation of stress fibers using FITC labeled phalloidon.

siRNA treated cells may also be examined for the expression of CD31 antigen, a marker for neoangiogenesis by ELISA, western blot, as well as immunoflourescence assays. The siRNA treated cells may also be analyzed for their ability to prevent wound healing using dermal endothelial cells in wound healing assays.

### Effect On mRNA And Pprotein Levels

Cells transfected with the siRNA may also be analyzed for reduction in levels of specific mRNAs using real time quantitative PCR analysis or Northern blot analysis. In real-time PCR, the preparation of the first strand cDNA is carried out using kits from Qiagen. The first strand cDNA from siRNA, negative RINA (i.e., scrambled siRNA, such as purchased from Ambion Inc.) and untreated samples is used as templates and mRNA quantity is detected by normalizing against internal control. The fold change in mRNA levels is determined by the protocol of Kenneth and Thomas (“Analysis of relative gene expression data using real-time quantitative PCR and 2^{-ΔΔCt} method,” Methods 2001; 25: 402-408). See Table 6 below. The siRNA treated cells are analyzed for the gene expression levels of CD31 antigen by quantitative Real time PCR.

### Protein

Expression levels of VEGF-C, VEGF-A and RhoA were analyzed by Western blot analysis to determine knockdown levels. Although siRNA transfections exhibited reduced mRNA levels and reflect gene expression levels, the determination of protein levels established that the RNA acted as an siRNA rather than an miRNA. miRNA are short RNA molecules (e.g., 23 mer in length), where contiguous sequence do not exactly match the mRNA sequence of a target protein. miRNA bind to mRNA and prevent translation but do not cause mRNA degradation. By contrast, when using siRNA, the siRNA complementary binding to a target causes degradation of mRNA and thus causes translation repression. Hence, when using siRNA, mRNA levels also go down. The effect of thrombin on induction of RhoA levels and efficiency of siRNA in its inhibition may be determined by GST-RBD pull down assays.

### siNA Compositions

The present siNA may be used with or without additional factors. The present invention also provides cholesterol conjugated siNA of VEGF-C and RhoA complexed with PEI. The siNA molecules can also be chemically modified by introduction of a 2′-O-Methoxy modification and thus made nuclease resistant. The serum stability of these molecules may also be studied.

### Animal Models And Treatment

Mice, rat or rabbit may be subjected to laser degeneration of Brunches membrane and therefore angiogenesis is induced. In such assays, siRNA is applied either by intravitrial, periocular or intravenous routes, and the efficacy of these is tested for inhibition of neoangiogenesis. Nuclease resistant or unmodified siNA is administered by intravitrial, periocular, intravenous, or retroorbital routes to derive the benefits of anti-angiogenic properties in treating various ocular disorders. The siRNA is complexed or encapsulated in liposomes, and is applied to treat neoangiogenic, glaucoma and other ocular disorders which include age related macular degeneration, diabetic retinopathy and glaucoma apart from other neoangiogenic diseases.

The following examples demonstrate certain embodiments of the invention. It will be appreciated by those of skill in the art that the techniques disclosed in the examples represent techniques discovered by the inventors to function well in the practice of the invention, and thus can be considered to constitute examples of modes for its practice. However, those of skill in the art will, in light of the present disclosure, appreciate that changes may be made in the disclosed embodiments and still obtain a like or similar result without departing from the spirit and scope of the invention.

### EXAMPLE 1

### Design of 21, 22, 23, And 27 Mer siIRNAs Ffor Modulation of RhoA And VEGF-C Gene Expression

### Identification of Target Sites

Based on the literature of Henshel, A et al., “DEQOR: A web based tool for the design and quality control of siRNAs,” Nucleic Acids Res. 2004; 32: W113-W120; Ui-Tei, K, et al., “Guidelines for the selection of highly effective siRNA sequences for mammalian and chick RNA interference,” Nucleic Acid Res. 2004; 32 (3): 936-48; Sui, G., et al., “A DNA vector based RNAi technology to suppress gene expression in mammalian cells,” Proc. Natl. Acad. Sci USA 2002; 26 (2): 199-213; Kim, D. H., et al., “Synthetic dsRNA dicer-substrates enhance RNAi in plasmacytoid dendritic cells through TLR7,” Nature Medicine 2005; 11: 263-270; and Judge, A. D., et al., “Sequence dependent stimulation of the mammalian innate immune response by synthetic siRNA,” Nat. Biotechnol. 2005; 23 (4): 457-62, 21, 23 or 27 mers siRNAs were designed.

The following basic requirement were met when designing siRNA:

For designing 21 mer siRNAs:

1. All siRNA has GC content between 30-50%.

2. 3′- of each siRNA has a over hang of dTdT.

For designing 22 & 23 mer siRNAs:

1. All siRNAs start at 5′- either with G/C.

2. 3′- of each siRNA strand has a over hang of dTdT.

3. GC content of duplex is between 40-50%.

4. At least 5 A/U bases in the first 7 bases of 5′-terminal strand of antisense strand.

For designing 27 mer siRNAs:

1. GC content of duplex is between 40 -55%.

2. Sense strand is 25 nucleotides, where as antisense strand is always 27 nucleotides resulting in overhang at 3′- of antisense strand.

3. Last 2 nucleotides of 3′-sense strand contains deoxy sugar instead of ribosugar backbone.

4. 5′- of sense strand contains overhang, while 3′- is blunt ended.

### Target Site

The sequence of VEGF-C and RhoA genes were screened for accessible sites that could meet the above-mentioned criteria using various online available algorithms, as well as manually. Based on these and other criteria, the following target sequences were chosed to create siRNA to VEGF-C and RhoA.

TABLE 1
Target ORF sequences of VEGF-C and RhoA for siRNA synthesis

| [symbol] A | | | | |
|---|---|---|---|---|
| [symbol] plex | | | Start | End |
| [symbol] lecule | Gene ID | Target Sequence in ORF | site | site |
| [symbol] A 6 | NM_005429 | 5′-TGTACAAGTGTCAGCTAAG-3′ | 669 | 687 |
| | (VEGF-C) | (SEQ ID NO: 1) | | |
| [symbol] A 17 | NM_005429 | 5′-GAACCATGTGGATAACTTTAC-3′ | 1816 | 1836 |
| | (VEGF-C) | (SEQ ID NO: 2) | | |
| [symbol] A 30 | NM_005429 | 5′-GCACGAGCTACCTCAGCAAGACGTT-3′ | 960 | 984 |
| | (VEGF-C) | (SEQ ID NO: 3) | | |
| [symbol] A 50 | NM_001664 | 5′-CCTGAAGAAGGCAGAGATATGGCAA-3′ | 697 | 721 |
| | (RhoA) | (SEQ ID NO: 4) | | |
| [symbol] A 51 | NM_001664 | 5′-GACCAAAGATGGAGTGAGAGAGGTT-3′ | 762 | 786 |
| | (RhoA) | (SEQ ID NO: 5) | | |
| [symbol] A 52 | NM_001664 | 5′-GAATTAGGCTGTAACTACTTTATAA-3′ | 1172 | 1196 |
| | (RhoA) | (SEQ ID NO: 6) | | |

[symbol]

indicates data missing or illegible when filed

### EXAMPLE 2

### Preparation of siNA Molecules

The siNA molecules were synthesized by chemical means, employing commercially available machinery. Chemical methods were classified based on the type of protecting group incorporated at 2′-carbon position of the ribose sugar:

1. 2′-t-butyldimethylsilyl (TBDMS)

2. 2′-O-triisopropylsilyloxymethyl (TOM)

3. 2′-acetoxyethoxy chemistry (ACE)

The cycle began with the 3′-most nucleoside attached to solid support material or bead. The second nucleotide was coupled to the 5-hydroxyl of the first nucleoside. Capping prevented effectively the propagation of failed or short nucleosides. The internucleotidic phosphate bond was then oxidized to the final P(V) state. Finally, the 5′-protecting group on the new nucleotide was removed and the growing oligonucleotide was ready for addition of the next nucleotide. Once the nucleic acid molecule reached the desired length, it was further deprotected, cleaved from the solid support, and analyzed for purity and yield.

### Purification

The siRNAs were purified by desalting, followed by PAGE (polyacrylamide gel electrophoresis), or by IE-HPLC (Ion Exchange—High Performance Liquid Chromatography). The quality of each RNA strand was analyzed by MALDI-TOF and the yield was determined by integrated spectrophotometer absorbance at 30 nm. During quality control by MALDI-TOF, a difference of 4 atomic mass units is the maximum allowed difference from that predicted. After obtaining comparable yields for each strand as determined by absorbance at 30 nm, sense and antisense strands were annealed, and vacuum lyophilized. During experimentation with the siRNAs, the lyophilized powders were suspended in RNA suspension buffer consisting of 100 mM KCl, 30 mM HEPES buffer (pH 7.5), and 1 mM MgCl₂, and heated for 1 min at 90° C. and the incubated at 37° C. for 1 h to dissolve the lyophilized powder. By following these manufacturing protocols, the following siRNA having different 3′-end modifications and lengths were synthesized (Table 2).

TABLE 2
siRNA synthesized with end modifications for
VEGF-C and RhoA genes.

| [symbol] RINA | |
|---|---|
| Duplex | Duplex sequence with overhangs |
| [symbol] 06 SENSE | 5′-UGUACAAGUGUCAGCUAAGdTdT-3′ (SEQ ID NO: 7) |
| ANTISENSE | 5′-CUUAGCUGACACUUGUACAdTdT-3′ (SEQ ID NO: 8) |
| [symbol] 17 SENSE | 5′-GAACCAUGUGGAUAACUUUACdTdT-3′ (SEQ ID NO: 9) |
| ANTISENSE | 5′-GUAAAGUUAUCCACAUGGUUCdTdT-3′ (SEQ ID NO: 10) |
| [symbol] 30 SENSE | 5′-GCACGAGCUACCUCAGCAAGACGdTdT-3′ (SEQ ID NO: 11) |
| ANTISENSE | 5′-AACGUCUUGCUGAGGUAGCUCGUGCUG-3′ (SEQ ID NO: 12) |
| [symbol] 50 SENSE | 5′-CCUGAAGAAGGCAGAGAUAUGGCdAdA-3′ (SEQ ID NO: 13) |
| ANTISENSE | 5′-UUGCCAUAUCUCUGCCUUCUUCAGGUU-3′ (SEQ ID NO: 14) |
| [symbol] 51 SENSE | 5′-GACCAAAGAUGGAGUGAGAGAGGdTdT-3′ (SEQ ID NO: 15) |
| ANTISENSE | 5′-AACCUCUCUCACUCCAUCUUUGGUCUU-3′ (SEQ ID NO: 16) |
| [symbol] 52 SENSE | 5′-GAAUUAGGCUGUAACUACUUUAUdAdA-3′ (SEQ ID NO: 17) |
| ANTSENSE | 5′-UUAUAAAGUAGUUACAGCCUAAUUCAC-3′ (SEQ ID NO: 18) |

* Scrambled RINA used as negative control = “negative RINA” in this and other experiments presented below. The scrambled “negative” RINA is a commercially available negative control from Ambion Inc. [symbol]
indicates data missing or illegible when filed

### EXAMPLE 3

### VEGF-C And RhoA Expression Analysis By Reverse Transcriptase PCR

HUVEC (Human Umblical Vascular Endothelial Cells, ATCC) and PC3 (Prostate cancer cells, ATCC) cell lines were cultured in 5% CO₂ at 37° C. following instructions from ATCC. Cells at 60-70% confluence were subjected to total RNA isolation followed by first strand cDNA preparation using the Qiagen Fast lane cell cDNA kit with minor modifications. Briefly, 20,000 cells were pelleted and washed once with buffer FCW (Qiagen). Cells were lysed for 15 min. at room temperature using buffer FCP (Qiagen).

Genomic DNA contamination was eliminated by the addition of gDNA wipeout buffer (Qiagen) by incubating at 42.5° C. for 30 min. First strand cDNA was synthesized by the addition of Quantiscript reverse transcriptase at 42.5° C. for 45 min. followed by incubation at 95° C. for 3 min. The first strand cDNA prepared was either used immediately for reverse transcriptase PCR or stored until further use at −20° C. First strand cDNA were amplified by PCR using the following primer sequences:

VEGF-C Forward Primer:
(SEQ ID NO: 19)
5′-AAAGAACCTGCCCCAGAAAT-3′
VEGF-C Reverse Primer:
(SEQ ID NO: 20)
5′-TGGTGGTGGAACTTCTTTCC-3′
VEGF-C Probe:
(SEQ ID NO: 21)
5′-6-FAM-AATCCTGGAAAATGTGCCTG-3′
RhoA Forward Primer:
(SEQ ID NO: 22)
5′-TATCGAGGTGGATGGAAAGC-3′
RhoA Reverse Primer:
(SEQ ID NO: 23)
5′-TTCTGGGGTCCACTTTTCTG-3′
RhoA Probe:
(SEQ ID NO: 24)
5′-6-FAM-CCATCGACAGCCCTGATAGT-3′

Amplified products were resolved over 2% agarose. Arrowheads in FIG. 1 indicate the amplicon of specific gene products obtained, showing that HUVEC and PC3 cells express both VEGF-C and RhoA. See FIG. 1, lanes 1 (PC3 VEGF-C), 3 (PC3 RhoA), 6 (HUVEC VEGF-C) and 8 (HUVEC RhoA).

### EXAMPLE 4

### Oligonucleotide Transfections/siRNA Transfections

HUVEC cells (human umbilical vascular endothelial cells), HeLa (cervical cancer), PC-3 (prostate cancer), HTB-38 (colorectal carcinoma) and ARPE-19 (normal diploid retinal pigmented epithelial cells) cell lines were obtained from ATCC and were maintained at 70-80% confluence, with a change of medium 24 h prior to transfection in T-25 flasks. Cell lines were used for all transfections of siRNA before reaching passage number ten unless otherwise mentioned. At the time of transfection, cells were trypsinized and reseeded into either a 24-well plate or any other standard tissue culture disposable plasticware at appropriate cell density. Unless otherwise stated, all transfections were carried-out in a 24-well plate with varying cell densities depending on cell lines used for a given experiment. Each well of a 24-well plate is seeded with appropriate cell densities one hour prior to transfections with growth medium not exceeding 400 μL and incubated in a 37° C. incubator with 5% CO₂. To this medium, diluted siRNA were added to a final concentration of 10 nM (in 97 μL of Opti-MEM I added 0.3 μL of siRNA from a 20 μM stock). To the diluted siRNA, 3 μL of Hiperfect transfection agent (Qiagen) was added and mixed by vortexing before incubating at room temperature for 10 min. For combination of siRNAs, such as VEGF-C and RhoA siRNAs, individual siRNA were mixed 10 nM each and used. All experiments also included a negative negative RINA obtained from Ambion.

All siRNA and transfection mixes were performed as master mixes from which appropriate volumes were added to the wells seeded with cells. At the end of incubation, siRNA-liposome complexes were mixed thoroughly and gently added dropwise to each well. The wells of a 24-well plate were mixed to uniformity and the plates incubated for appropriate incubation times in 37° C. CO₂ incubators for further analysis of cells. Transfection efficiencies are obtained for each cell line by counting number of cells showing Cy3-labeled siRNA (using negative negative RINA from Ambion) after 16 h of transfection. After 16 h of transfection, cells were trypsinised and washed once in PBS and suspended in the same. Cells that were suspended in PBS were observed with an inverted fluorescent microscope and were counted for the number of fluorescent labeled cells and total number of cells in 15 different fields of microscope each field. The percentage of cells that were labeled with Cy3 siRNA was determined and thus transfection efficiency was derived. Of all the cell lines tested, HeLa gave 97% transfection efficiency, whereas ARPE-19 gave only 85% efficiency, as shown in Table 3.

TABLE 3
Percent of Transfection efficiencies as determined by Cy3 labeled
siRNA for different cell lines.

| Cell line transfected | % of Transfection |
|---|---|
| HUVEC | 95 ± 6.0 |
| HeLa | 97 ± 5.0 |
| PC3 | 85 ± 3.0 |
| ARPE-19 | 85 ± 5.0 |
| HTB-38 | 90 ± 9.0 |

### EXAMPLE 5

### VEGF-C And RhoA siRNAs Inhibit VEGF-A Secretion

### A) Inhibitory Effect of VEGF-C And RhoA On VEGF-A Secretion

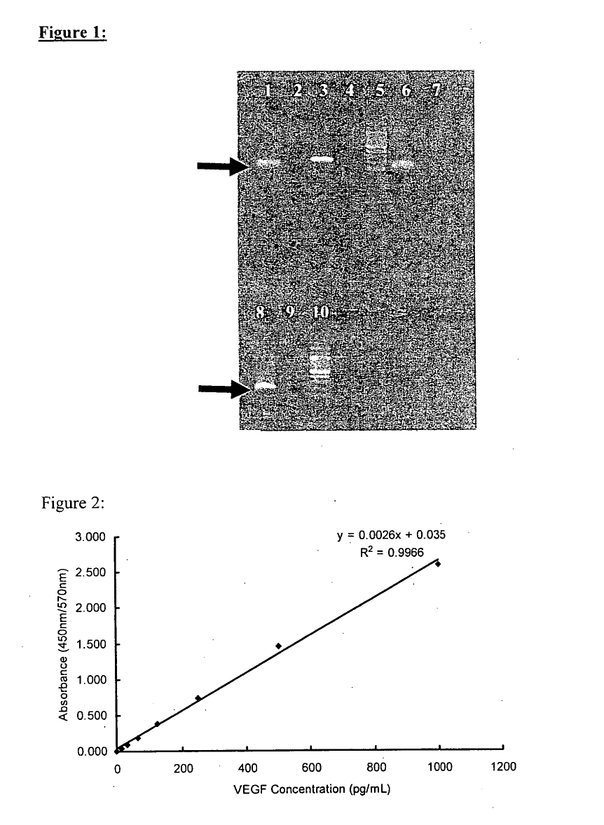

PC3 (prostate cancer), ARPE-19 (retinal pigmented epithelial cells), HeLa (cervical cancer) and HCC-38 (breast cancer) cells were transfected with 10 nM siRNA, i.e., one of six siRNAs (RINA 6, 17, 30 against VEGF-C, and RINA 50, 51, 52 against RhoA), using HiPerFect Transfection Reagent following protocol of manufacturer (Qiagen). At the end of 72 h of transfection cell, supernatants were analyzed by Sandwich ELISA as per the instructions of VEGF-A ELISA kit (Calbiochem). A standard curve was obtained for concentrations (15.6 pg/mL to 1000 pg/mL) of VEGF-A, as shown in FIG. 2.

The quantity of secreted VEGF-A in supernatants was estimated from the standard curve for VEGF-A (FIG. 2). All tested VEGF-C siRNAs (RINA 6, 17 and RINA 30) caused inhibition of VEGF-A secretion in two or more cell lines (PC3, ARPE-19 and/or HeLa) upon transfection. See Table 4 below. Of the three VEGF-C siRNAs tested, RINA 30 transfection of HeLa cells showed the greatest effect. RINA 30 inhibited secretion of VEGF-A by 68% in HeLa, as compared to mock (negative RINA) treated cells.

TABLE 4
Knockdown of VEGF-C inhibits VEGF-A secretion (165 kDa soluble
form of VEGF-A secreted in supernatant, as detected by ELISA)
VEGF-A secreted in pg/mL*

| siRNA | PC3 | ARPE-19 | HeLa |
|---|---|---|---|
| RINA 6 | 842.76 ± 0.28 | 714.76 ± 0.85 | 435.33 ± 0.15* |
| RINA 17 | 918.6 ± 0.96 | 453.43 ± 0.01* | NA |
| RINA 30 | 775.76 ± 0.13* | 386.6 ± 0.15* | 300 ± 0.89* |
| Negative RINA | 1187.1 ± 0.75 | 705.6 ± 0.48 | 949.01 ± 0.67 |
| Untreated | 1452.16 ± 10 | 952.4 ± 21.3 | 1103.11 ± 11 |

*Indicates P ≦ 0.05 in comparison with negative RINA

Similarly, all tested RhoA siRNAs (RINA 50, 51 and RINA 52) caused inhibition of VEGF-A secretion in at least one cell line (ARPE-19, HeLa and/or HCC-38) upon transfection. Of the three RhoA siRNAs tested, RINA 50 showed the greatest effect. RINA 50 inhibited secretion of VEGF-A by 40% in HeLa cells, as compared to mock treated cells. RINA 52 inhibited secretion of VEGF-A by 27% in ARPE-19 cells.

TABLE 5
Knockdown of RhoA inhibits VEGF-A secretion
VEGF-A secreted in pg/mL*

| siRNA | ARPE-19 | HeLa | HCC-38 |
|---|---|---|---|
| RINA 50 | 712 ± 3.5 | 657 ± 4.5* | 388 ± 4.5* |
| RINA 51 | 938 ± 2.8 | N.A | N.A |
| RINA 52 | 687 ± 6.3* | N.A | N.A |
| Negative RINA | 930 ± 6.8 | 1103 ± 1.23 | 470 ± 6.2 |
| Untreated | 938 ± 5.2 | 1391 ± 0.24 | 466 ± 2.0 |

*Indicates P ≦ 0.05 in comparison with negative RINA

Results presented herein indicate that siRNAs directed to either VEGF-C or RhoA unexpectedly inhibit expression and secretion of VEGF-A. The results likewise indicate for the first time that inhibition of either VEGF-C or RhoA expression leads to an inhibition of VEGF-A expression, secretion and VEGF-A-specific cell signaling. Results will indicate that use of a VEGF-C siRNA and a Rho siRNA together inhibit VEGF-A expression and secretion in a synergistic manner, as compared to additive effects of using VEGF-C siRNA or RhoA siRNA alone; and more than direct inhibition of VEGF-A

### B) Quantitative Real Time PCR Analysis Expression Levels of Target Gene (RhoA And VEGF-C) mRNA

The expression levels of VEGF-C and RhoA genes was determined by Real time PCR. The cell lines used in this study included PC3 (prostate cancer) HeLa (cervical cancer), ARPE-19 (retinal pigmented epithelial cells) and HTB-38 (colorectal cancer) obtained from ATCC. Cells were transfected either with 10 nM of RINA 52 or RINA 30 and negative negative RINA. At the end of 72 h of post transfection, the first strand cDNA preparation was carried-out using Qiagen Fast lane cell cDNA kit with minor modifications. Briefly 20,000 cells were pelleted and washed once with buffer FCW (Qiagen). Cells were lysed for 15 min at room temperature using buffer FCP (Qiagen). Genomic DNA contamination was eliminated by the addition of gDNA wipeout buffer (Qiagen) by incubating at 42.5° C. for 30min. First strand cDNA was synthesized by the addition of Quantiscript reverse transcriptase at 42.5° C. for 45 min followed by incubation at 95° C. for 3 min. The first strand cDNA prepared was either used immediately for quantitative Real time PCR or stored till further use at −20° C. Real time quantitative PCR can be accomplished following standard protocols and using commercially available machines. First strand cDNA from antisense, negative RINA and untreated samples were used as template, and the levels of mRNA was quantified by normalizing against the internal control β-actin. The expression of RhoA and VEGF-C was determined as a percent decrease in expression level over untreated cells as indicated in Table 6.

TABLE 6
Percent decrease in expression levels of target gene at 72 h post
transfection as determined by Real Time PCR*

| Cell-line | | Negative RINA | Untreated |
|---|---|---|---|
| | RhoA | | |
| HeLa | 96.50 | −1.0 | 0 |
| HTB-38 | 96.30 | NA | 0 |
| PC3 | 73.82 | 6.0 | 0 |
| | VEGF-C | | |
| ARPE-10 | 85.0 | 5.0 | 0 |
| PC3 | 87.0 | NA | 0 |

Quantitative real time PCR analysis of RINA 52 transfected cells shows a 96% decrease in mRNA levels of RhoA gene in HeLa and HTB-38 cells, as compared to mock transfected cells. RINA 30 transfection resulted in a decrease in mRNA levels of gene VEGF-C by 87% and 85% respectively in the case of PC3 and ARPE-19 cells.

### C) Determination of Inhibitory Concentration 50 (IC50) Values For RINA 30 Transfected MCF-7 Cells For VEGF-C And VEGF-A By Sandwich ELISA

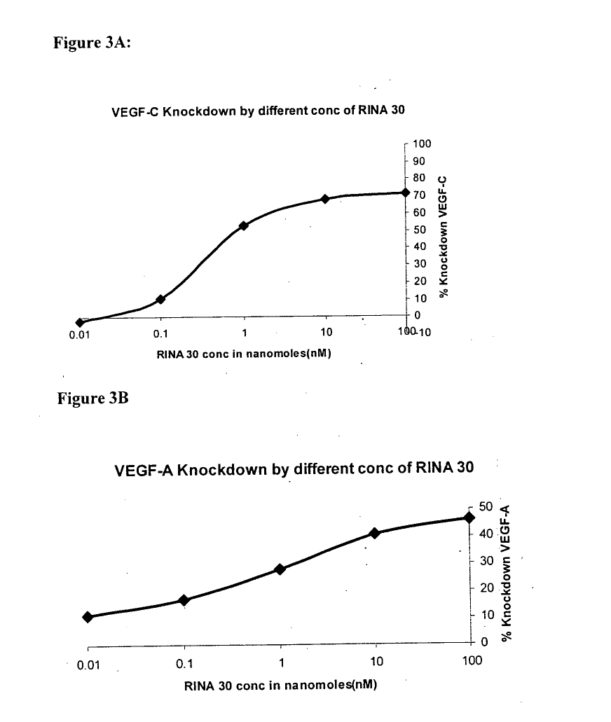

Breast cancer cell lines (MCF-7) were transfected with varying concentration of VEGF-C RINA 30 (0.01, 0.1, 1.0, 10.0, and 100.0 nM) or with negative RINA. At the end of 72 h of transfection, cell culture supernatants were clarified from respective wells and subjected to Sandwich ELISA to determine the level inhibitory levels for VEGF-C cell expression, as well as VEGF-A expression. VEGF ELISA kit (Human,Cat.no QIA51, CALBIOCHEM) was used for VEGF-A, and Quantikine VEGF-C (Human, kit R&D systems, Cat. no. DVEC00) was used for VEGF-C. With regard to VEGF-C expression (as compared negative RINA transfected cells), RINA 30 reached a plateau at 70% knockdown of VEGF-C expression at 10 nM concentration, and exhibited 50% knockdown at 0.8 nM, as shown in FIG. 3A. VEGF-A reached a plateau of 48% knockdown of VEGF-A at 10 nM concentration of RINA 30, while its IC50 value remained at 0.8 nM, as shown in the FIG. 3B. The IC50 values and Real time PCR data show that RINA 30 is highly potent and works to inhibit VEGF-C and VEGF-A expressions at 0.8 nM. The results presented herein show that expression of VEGF-C regulates expression levels of VEGF-A, indicating that there exists a homeostasis and that both VEGF-C and VEGF-A are required for neoangiogenesis.

### D) Analysis of VEGF-C Protein Levels By Western Blot

Cells (Hela, PC3, MCF-7, HTb-38, ARPE-19 and HUVEC) are transfected with RINA 6, 17 and 30. At the end of 72 h of transfection, protein lysates are obtained employing

Mammalian protein extraction reagent, MPER (Pierce) and are subjected to Western blot analysis. The protein knockdown levels are detected from the analysis of protein blots. VEGF-C expression levels are inhibited in the cells upon transfection with RINA 6, 17 and/or 30, as compared to negative RINA transfected cells.

### E) Analysis of RhoA Protein Levels By Western Blot

Cells (Hela, PC3, MCF-7, HTb-38, ARPE-19 and HUVEC) are transfected with RINA 50, 51 and 52. At the end of 72 h of transfection, protein lysates are obtained employing Mammalian protein extraction reagent, MPER (Pierce) and are subjected to Western blot analysis. The protein knockdown levels are detected from the analysis of protein blots. RhoA expression levels are inhibited in the cells upon transfection with RINA 50, 51 and/or 52, as compared to negative RINA transfected cells.

### EXAMPLE 6

### Knockdown of RhoA Inhibits Phosphorylation of ROCK

Rho Kinase 1 (ROCK-1) and Rho kinase 2 (ROCK-2) are Ser/Thr kinases that are activated by RhoA and act as effector molecules of RhoA, resulting in cytoskeletal reorganization. This reorganization results in endothelial cell morphogenesis leading to formation of new blood vessels.

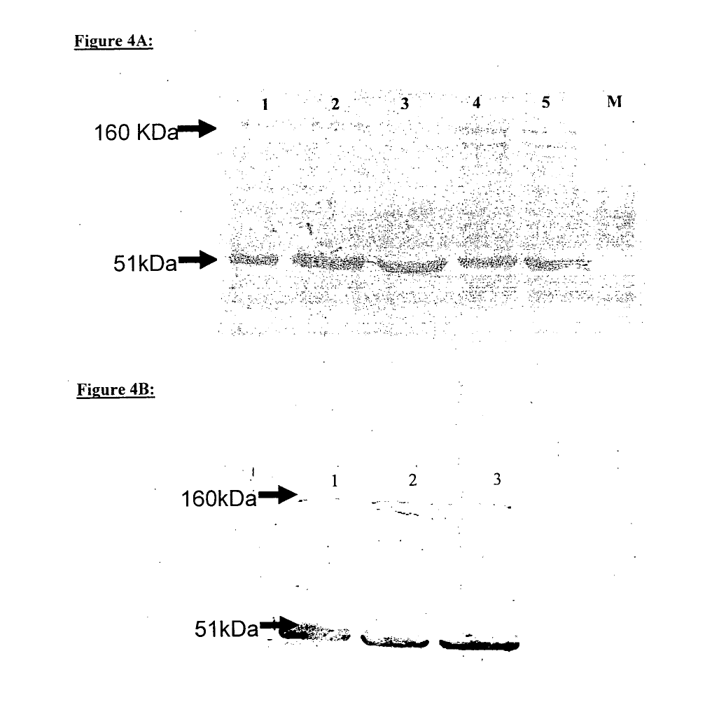

Knockdown of RhoA and its effect on activation of Rho kinases was determined. Cells (ARPE-19) were transfected with RINA 50, 51 or 52 and analyzed for protein expression or phosphorylation status of ROCK-1 or ROCK-2 at the end of 72 h of transfection (FIGS. 4A and 4B). Protein lysates were made using mammalian protein extraction reagent (MPER, Calbiochem) following manufactures protocol. Based on Bradford total protein estimations, equal quantities of proteins were resolved over 10% SDS PAGE. Proteins resolved over the SDS-PAGE were subjected to Western blot transfer at 110 V for 70 min. on to a pre-wet nitrocellulose membrane along with pre-stained rainbow molecular weight markers (Amersham Biosciences). The transfer of proteins by electro-blotting was confirmed by Ponceau S staining (Sigma). The blot was incubated in blocking solution (5% skim milk powder) for 1 h at room temperature on a rocking platform. Before incubating with an anti-ROCK-1 mouse monoclonal IgG1 (Santa Cruz) or an anti-phospho-ROCK-2 antibody ABCAM ab 24843 and mouse alpha tubulin antibody (Sigma) as internal control, the blot was washed over an orbital shaker for 5 min, each with change of PBST (phosphate buffered saline containing 0.1% Tween 20). The blot was incubated with primary antibody overnight at 4° C. After washing with PBST to remove any non-specific bound primary antibodies, the blots were incubated with secondary antibodies conjugated with alkaline phosphatase for two hours at room temperature over an orbital shaker. Secondary antibodies included rabbit anti-mouse antibody conjugated with alkaline phosphatase (SIGMA) to detect tubulin and Rock-1, and goat anti-rabbit antibody (SIGMA) to detect ROCK-2 phospho-antibody. In FIG. 4A, primary antibodies were Rabbit anti phospho Rho kinase alpha(Rock-2) antibody (Abcam, Cat. No. ab24843.2.), Mouse Anti alpha Tubulin Antibody (SIGMA Cat. No. T6199). Secondary antibodies were rabbit anti-mouse antibody(gamma chain specific) conjugated with alkaline phosphatase (SIGMA Cat. No. A3438); Goat anti Rabbit antibody (Whole molecule) conjugated with alkaline phosphatase (SIGMA Cat no A3687).

In FIG. 4B, the primary antibodies are Anti ROCK-1 mouse monoclonal IgG1 (Santa Cruz Cat. No. SC17794) and Mouse Anti alpha Tubulin Antibody, (SIGMA Cat. No. T6199); while the secondary antibodies was rabbit anti-mouse antibody (gamma chain specific) conjugated with alkaline phosphatase (SIGMA Cat. No. A3438). Blots were washed three times with PBST for 10 min each before being developed with BCIP/NAT substrate solution (SIGMA). Protein bands corresponding to phosphorylated ROCK-2 (160 KDa) and tubulin (51 Kda) as endogenous control were detected, as shown in the FIG. 4A. Protein bands corresponding to ROCK-1 (160 KDa) and tubulin (51 Kda) as endogenous control were detected, as shown in the FIG. 4B. RINA 52 caused the greatest inhibition of ROCK-2 phosphorylation among the three siRNAs (RINA 50, 51 and 52) tested in this experiment. See lane 3 in FIG. 4A.

The results presented here show for the first time that siRNAs directed to RhoA inhibit ROCK phosphorylation (as demonstrated for ROCK-2 here) in relevant cells, such as retinal pigmented epithelial cells. Thus, siRNAs of the present invention inhibit the ROCK signaling pathway, without affecting expression of ROCK itself as seen in regard ROCK 1 in FIG. 4B. This is in contrast to results expected when using a siRNA directed to ROCK itself, which presumably affects gene expression of ROCK.

### EXAMPLE 7

### RINA 30 And RINA 52 Do Not Induce An Interferon Response Upon Transfection Into ARPE-19 Cells

Retinal pigmented cells were transfected with 10 nM VEGF-C siRNA (RINA 6 and 30) and RhoA siRNA (RINA 50, 51 and 52). At the end of 20 h of transfection, cells were lysed and the otal RNA was prepared. Interferon response pathway specific gene expression was determined for the following genes following the manufacturers instructions from the Interferon Response Detection Kit for validation of siRNA experiments (SBI): Interferon response genes OAS1(NM_{—}016816) and OAS2 (NM_{—}016817.1) represents 2′,5′-oligoadenylate synthetase (OAS); MX1 (NM_{—}002462.2) represents Myxovirus (Influenza virus) resistance protein family; and IFITMI (NM_{—}003641.2) represents interferon inducible trans-membrane proteins. Of the siRNAs tested, RINA 30 and 52 caused only a low level enhancement of expression with regard to genes MX1 or ISGF3γ, respectively, and caused little to no measurable enhancement of expression of other genes involved in eliciting an interferon response. See Table 7. Thus, results presented herein demonstrate that cellular responses observed upon transfection with siRNAs of the present invention, such as VEGF-C RINA 30 and RhoA RINA 52, are not due to an activation of an interferon response, but rather are due to, inhibition of expression of the specific gene(s) of interest.

TABLE 7
Lack of interferon response induced by RINA 30 and 52, as seen in
reverse transcriptase PCR analysis
ARPE-19 Interferon response as determined by RT-PCR

| | | | | | | |
|---|---|---|---|---|---|---|
| Gene | UT | 6 | 30 | 50 | 51 | 52 |
| OAS1 | + | + | + | ++ | + | + |
| OAS2 | + | + | + | + | + | + |
| ISGF3γ | + | ++ | + | +++ | +++ | ++ |
| MX1 | + | ++ | ++ | +++ | + | + |

NOTE:
UT = Untreated
+ Indicates levels in untransfected.
++ Indicates observable enhancement.
+++ Indicates prominent unambiguous enhancement.

### EXAMPLE 8

### In Vitro Angiogenesis Assay

Human umbilical vein endothelial cells (HUVEC) obtained from ATCC were cultured in endothelial cell culture medium following directions from ATCC. HUVEC cells were transfected individually with 5 nM of RINA 6, 30, 50, 52 and negative RINA. At the end of 48 h of transfection, cells were trypsinized and plated on ECM (extracellular matrix) coated wells of a 96-well plate in triplicate, each at a concentration of 5000 cells per well. Cells seeded onto ECM coated 96-well plates were cultured and observations were made under light microscope for the following parameters to be quantified at the end of 8 h of incubation on the ECM:

A) Migration of HUVEC cells to close proximity of each other.

B) Alignment of cells in the form of a vessel or tube.

C) Formation of vascular sprouts.

D) Establishment of closed polygons form.

E) Formation of complex mesh like structures.

Comparisons were made between untreated, negative RINA treated and RINA treated wells in duplicate. The number of sprouts or vessels present in each well was quantified at five different fields from duplicates. For example, FIG. 5 shows angiogenesis in untreated HUVEC cells (FIG. 5A) or cells treated with negative RINA (FIG. 5B), with arrows showing vessel branching. In FIG. 5C, by contrast, HUVEC cells treated with RINA30 show cells separated from each other, and exhibited no signs of cell migration or initiation of vessel formation.

Results herein show that transfection of HUVEC cells with VEGF-C or RhoA siRNA caused an inhibition of the formation of vessels, as compared to that seen in mock or untreated cells. Of all siRNAs tested, RINA 30 and 52 exhibited maximum inhibition of the vessels formation. In addition, no vessel formation was noted even after 16 h after plating on ECM in cells transfected with both VEGF-C RINA 30 and RhoA RINA 52. These results indicate that knockdown of either RhoA or VEGF-C inhibits vessel formation and that this inhibition is durable, as shown in Table 8.

TABLE 8
Percent of angiogenesis inhibition over untreated samples as obtained
from light microscopic observations post 8 h of incubation on
extracellular matrix (ECM)

| RINA | % of Angiogenesis | Std |
|---|---|---|
| 6 | 13.04* | ±2.0 |
| 30 | 0* | ±0.0 |
| 50 | 13.04* | ±1.2 |
| 52 | 6.52* | ±3.0 |
| 30 + 52 | 0* | ±0.0 |
| Negative RINA | 80.10 | ±10.0 |
| UT | 100 | ±7.5 |

*Indicates P ≦ 0.05 in comparison with negative RINA

### EXAMPLE 9

### Effect of Knockdown of VEGF-C And RhoA On Cytokine Profile In ARPE-19 Cells

Retinal pigmented epithelial cells are transfected with RINA 30, 52 or their combination. At the end of 72 h of transfection, cell supernatants are obtained from the RINA treated, negative RINA treated and untreated cells. The supernatants are analyzed for 28 different cytokines following the protocol of Human Cytokine Array Panel A Array kit from R & D systems. The implications of change in expression profiles of various cytokines in relation to angiogenesis is obtained. Most of the pro-angiogeneic cytokines are inhibited to various degrees, while anti-angiogenic cytokines are over-expressed.

### EXAMPLE 10

### Transcriptome Analysis of VEGF-C And RhoA Knockdown In Retinal Pigmented Epithelial Cells

To test the specificity of the different VEGF-C and RhoA siRNAs, retinal pigmented epithelial cells (ARPE-19) are transfected with RINA 30, 52 or their combination as described earlier. At the end of 72 h of transfection, total RNA is prepared following the protocol of Qiagen total RNA isolation kit (RNeasy Mini kit). Total RNA of 2 μg is suspended in 10 μL of water. The quality of RNA is checked on denaturing formaldehyde gels and the OD ratio is determined using a Perkin Elmer Spectrophotometer. One μg of total RNA is converted into DIG labeled cRNA following the protocol of Nano In-vitro Transcription amplification kit from Applied Biosystems. Fourteen micrograms of cRNA is hybridized to Human Genome Survey arrays containing 60 base pair probes for interrogation of 29,098 genes. The arrays are hybridized at 55° C. for 17 h and subsequently washed and bound to antibody against DIG coupled with alkaline phosphatase. After the addition of chemiluminescent detection substrate, the arrays are scanned on the 1700 analyzer. Autogridding are performed by the imaging software and the result file is created that transforms the intensity of each gene into a numeric value, i.e., a higher the signal corresponds to a higher the numeric value, which corresponds to a higher amount of gene present in the sample

Controls are added for each and every step of the assay starting from reverse transcription (RT), in vitro transcription (IVT), hybridization and chemiluminescence detection. A quality report is generated for these controls that help to ascertain the success of the microarray experiment. A secondary analysis using Spotfire will then be performed. The Spotfire software normalizes the data and performs a “t test” to determine the differentially expressed genes between two conditions. It also averages the replicates and determines a fold change value for the two conditions. The probe IDs that are differentially expressed are sorted through bead studio 3.0 version of software and change in expression profile of various genes above 3 fold is obtained. Analysis of the change in gene expression profiles and their relevance to inhibition of angiogenesis process are obtained

### EXAMPLE 11

### Effect of RhoA, VEGF-A And VEGF-C siRNA On Cells Under Conditions of Hyperglycemia And Hypoxia

### Materials And Methods

### Cell Lines

The human cell lines RPE19 and MCF7 (American Type Culture Collection, Manassas, Va., USA) and endothelial cell lines, HUVEC and HMVEC (Lonza, Walkersville, Md., USA) were cultured in a humidified atmosphere of 5% CO₂ at 37° C. as per instructions. Sub-confluent cultures (60-70% confluent) not exceeding 10 passages were used in experiments. Endothelial cells were used at passages not exceeding 5-7.

siRNA Transfections

The mRNA sequences for human VEGF-C (Gene bank Accession No. NM_{—}005429) and human RhoA (Gene bank Accession No. NM_{—}001664) were used to design siRNA's targeting VEGF-C and RhoA respectively. Three double-stranded siRNAs for each targeting genes VEGF-C and RhoA were custom synthesized through Qiagen (Hilden, Germany), as per earlier Examples. Pre-validated siRNA 462 targeting VEGF-A was obtained from Ambion cat no (siRNA i.d s462) (Austin, Tex., USA). Transfections were performed with 10 nM siRNA (unless otherwise stated), using Hiperfect transfection reagent (Qiagen) as per the manufacturer's instructions. The transfection efficiency in cell lines was determined using Cy3 labeled control—siRNA (negative RINA was labeled with Cy3 using Silencer siRNA labeling kit-Cy3 from Ambion following manufacturer's instructions).

### VEGF ELISA

After 72 h post-transfection, the supernatants from 24-well plates were harvested and VEGF-A and VEGF-C ELISA was performed according to the manufacturer's instructions (R&D Systems, Minneapolis, Minn., USA). Data was obtained from triplicate wells and statistical significance was determined at P≦0.05 by student's t test.

### Immunoblotting

Protein lysates were prepared from siRNA-transfected cells (after 72 h) with Mammalian Protein Extraction Reagent (Pierce, Rockford, Ill., USA), and the protein content of the lysate was estimated using the Bradford reagent (Biorad, Hercules, Calif., USA). Equal amounts of protein were loaded onto SDS-PAGE and western blot analysis was carried out using monoclonal antibody to RhoA (sc-418, Santa Cruz Biotech., Santa Cruz, Calif., USA), as previous described (Suarez et al. “The role of VEGF receptors in angiogenesis; complex partnerships,” Cell Mol Life Sci 63: 601-6125 (2006)). Monoclonal antibody against a-tubulin (clone DM1A, T6199, Sigma) was used to detect a-tubulin, as an internal control. Goat anti-mouse IgG (γ-chain specific)-Alkaline phosphatase-(A3438, Sigma, St. Louis, Mo., USA) was the secondary antibody. All protein blots were repeated three times from independent transfections and their standard deviations (S.D) were determined from densitometric analysis.

### Quantitative Real Time PCR

First strand cDNA was prepared from siRNA transfected RPE19 and MCF7 cells using Fast cell cDNA kit (Qiagen) as per the manufacturer's instructions. Quantitative Real Time PCR was performed using the Applied Biosystems 7500 system (ABI, Foster city, CA, USA) and data analyzed as per manufacturer's instructions. mRNA abundance was measured by real-time quantitative PCR at 72 h post-transfection for screening siRNAs designed. The efficacy of siRNA was determined at 72 h post transfection. 13-actin was used as the internal reference control. All the experiments were repeated twice from independent transfections, each in triplicate.

### Dosage Studies

Cells lines (RPE19, MCF7 or HUVEC) were transfected with siRNA as described earlier, using five concentrations at one log increment each (0.01 nM, 0.1 nM, 1 nM, 10 nM and 100 nM). After 72 h, clarified cell supernatants were obtained and analyzed by ELISA to measure the concentration of VEGF-C or VEGF-A secreted from transfected cells.

Hypoxia experiments

For hypoxia experiments, the oxygen content was set to 10% with the loss of partial oxygen pressure replaced with nitrogen and CO₂. Cell lines were maintained under hypoxic conditions for 24 h prior to transfection, and 72 h post-transfection.

### Hyperglycemic Experiments

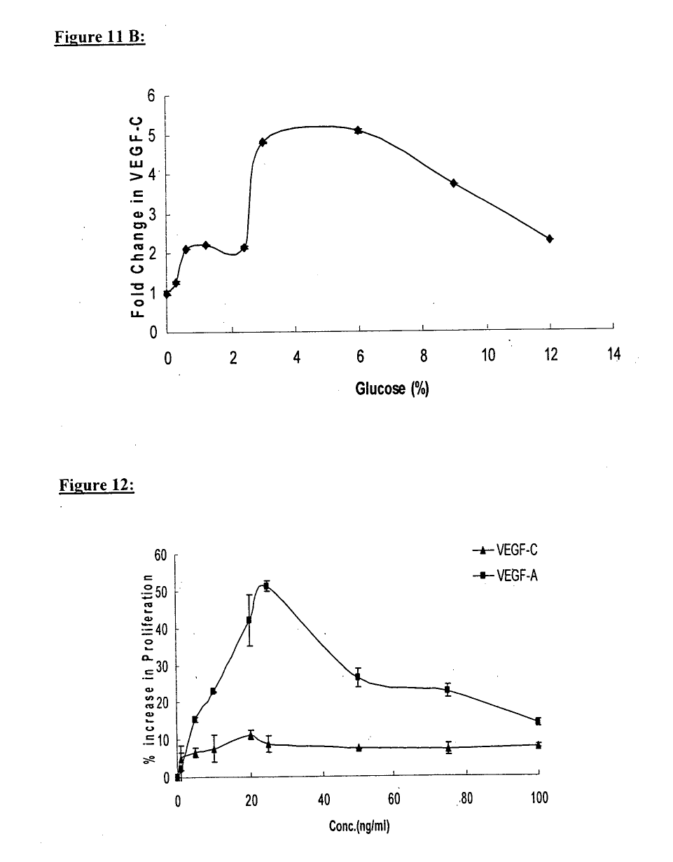

For hyperglycemia experiments RPMI1640 medium was supplemented with glucose at concentrations ranging from 0-12%, in increments of 2%. The optimum glucose concentration for VEGF-A or C secretion was determined by ELISA. (See FIGS. 11A and 11B).

### MTS Proliferation Assay

HUVEC cells were transfected with RINA52 or negative RINA at a concentration of 100 nM. After 24 h, the cells were trypsinised and seeded at a cell density of 3000 cells/well in a 96-well plate. This was followed by the addition of VEGF-A (Sigma), VEGF-C (Prospec), or their combination, at 5 ng·mL⁻¹. Cells with and without bovine brain extract was included as controls. After 48 h, proliferation was measured using the Cell Titer aqueous one solution cell proliferation assay (Promega Corp., Madison, Wis., USA) as per manufacturer's instructions.

### In Vitro Tube Formation Assay

In vitro tube formation was assessed using the in vitro angiogenesis assay kit of Chemicon (Temecula, Calif., USA) as per the manufacturer's instructions. Briefly, 72 h after transfection with RINA52, HUVEC or HMVEC cells were seeded on extracellular matrix and allowed to form a capillary tube. The culture medium was supplemented with growth factors such as VEGF-C or VEGF-A as, and where, applicable. Capillaries were observed at the end of 18 h from the time of seeding. Capillary tube widths, total lengths, and number of branch points was quantitated in three random fields, scores assigned, and the values averaged.

### Phalloidin Staining

The effect of down regulation of RhoA on stress fiber formation was determined as follows. Briefly, HUVEC, cells were transfected with RINA52 on coverslips, and after 72 h; the cells were fixed with 4% paraformaldehyde at room temperature for 20 min and then incubated with ice-cold 100% acetone at −20° C. for an additional 20 min. The fixed cells were incubated with a 1:200 dilution of Texas Red-X phalloidin (Molecular probes, Eugene, Oreg., USA) for 1 h at room temperature. The cover slips were mounted using Vectashield with DAPI (Vector Laboratories) and the cells were examined under Nikon Eclipse TE300 fluorescent microscope.

### Statistical Analysis

The error bars represent the mean±1 standard deviation (SD) and significance was calculated by two tailed student's t test assuming equal variance. Differences were considered significant at P≦0.05.

### Results

siRNA Transfection Efficiency

Cy-3 labeled Negative RINA at 10 nM was introduced into cells using Hiperfect transfection reagent. Delivery of siRNA by Hiperfect transfection reagent resulted in transfection efficiencies ranging from 87-98% (Table 9).

TABLE 9
Percent of Transfection efficiencies as determined by Cy3
labeled siRNA for different cell lines.

| Cell line transfected | % of Transfection |
|---|---|
| HUVEC | 95 ± 6.0 |
| ARPE-19 | 85 ± 5.0 |
| MCF-7 | 90 ± 7.0 |
| HMVEC | 96 ± 6.0 |

The transfection efficiencies obtained indicated that RPE19, MCF7, HUVEC and HMVEC cell lines were all efficiently transfected.

### VEGF-C Compensates Expression Levels of VEGF-A In Dosage Dependent Manner

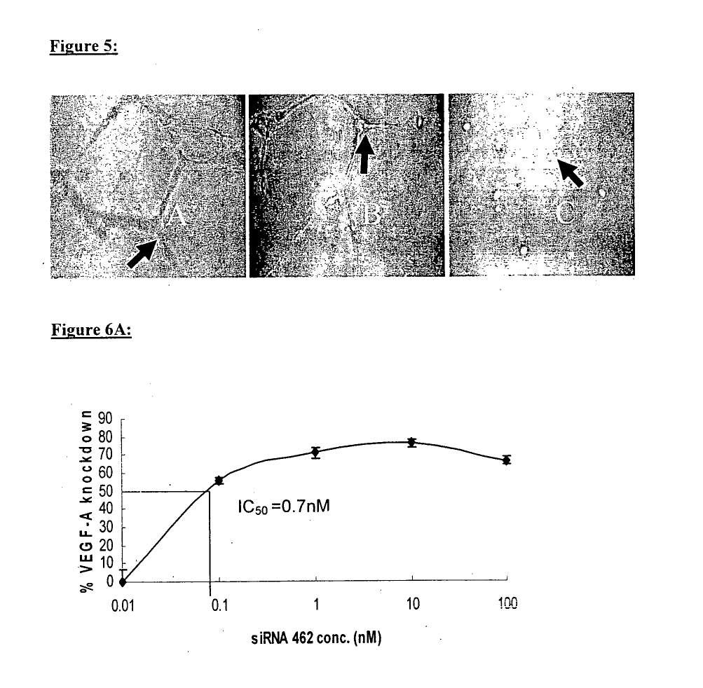

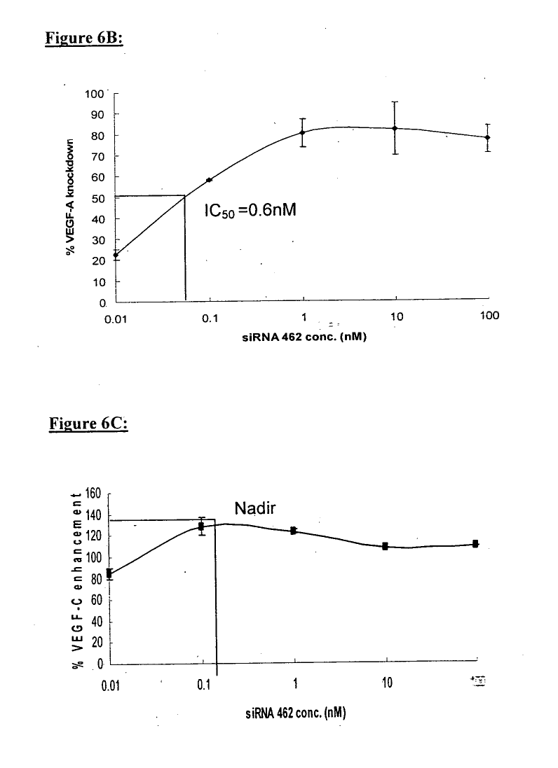

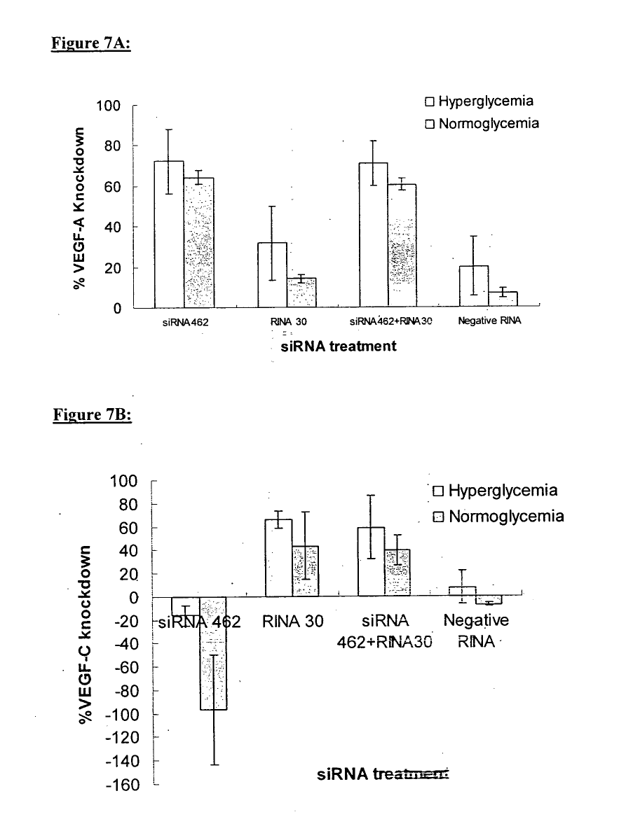

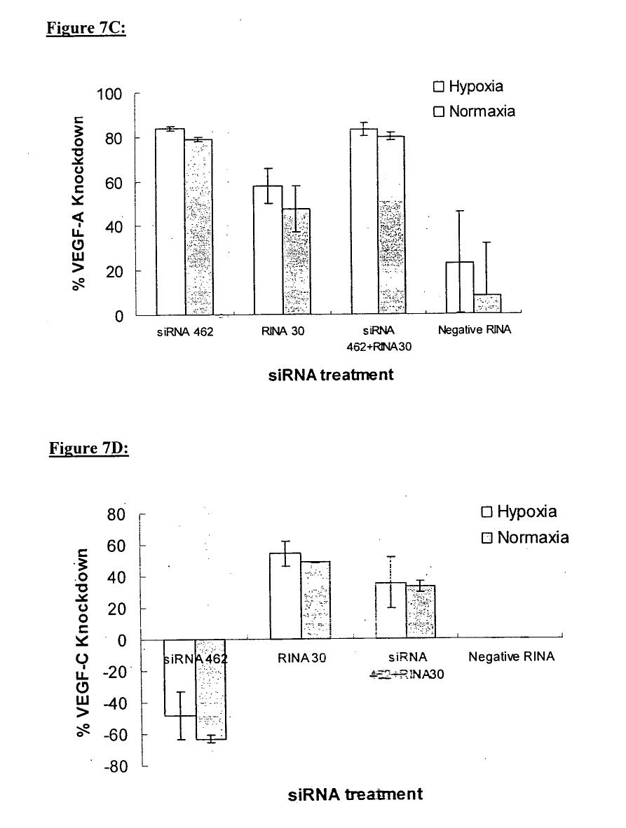

Previous experiments demonstrated that RINA30 and RINA52 showed highest efficacy in knocking down VEGF-C and RhoA, respectively, and did not elicit an interferon response. Knockdown of VEGF-A with siRNA462 caused decreased expression levels ranging from 0 to 70% for RPE19 (IC₅₀=0.7 nM, FIG. 6A) and 20 to 83% for MCF7 (IC₅₀=0.6 nM, FIG. 6B). Surprisingly, VEGF-A knockdown cells increased VEGF-C expression over negative RINA treated or untransfected cells in a dose-dependent manner. (FIG. 6C). Peak VEGF-C enhancement was observed at 60% VEGF-A knockdown in MCF7 cells (FIG. 6C). Thus, decreased VEGF-A expression results in increased VEGF-C expression. Similar results were observed under hyperglycemic as well as normoglycemic conditions in RPE19 cells (FIG. 7A-7B) while in MCF7 cells under hypoxic and normaxic conditions respectively (FIGS. 7C-D). Furthermore, in combination treatment (RINA30+siRNA462), expression levels of VEGF-A and VEGF-C are correlating with that of individual treatments in terms of knockdown obtained. (FIG. 7). The decrease in expression of VEGF-A and simultaneous increase in expression of VEGF-C were also observed at mRNA levels as determined by quantitative real time PCR (Table 5).

TABLE 5
VEGF-A and VEGF-C expression profile in
different cell lines transfected with siNA462 and siNA30.

| | VEGF-A | VEGF-C |
|---|---|---|
| ARPE19 | | |
| siNA462 | 66.14 ± 5.0 | +564.3 ± 46.5 |
| siNA30 | 47.0 ± 18.4 | 53.6 ± 5.4 |
| MCF-7 | | |
| siNA462 | 51.12 ± 6.67 | +116.0 ± 7.5 |
| siNA30 | 43.5 ± 9.2 | 65.4 ± 12.2 |

### Knockdown of VEGF-C Results In Decreased Expression Levels of VEGF-A

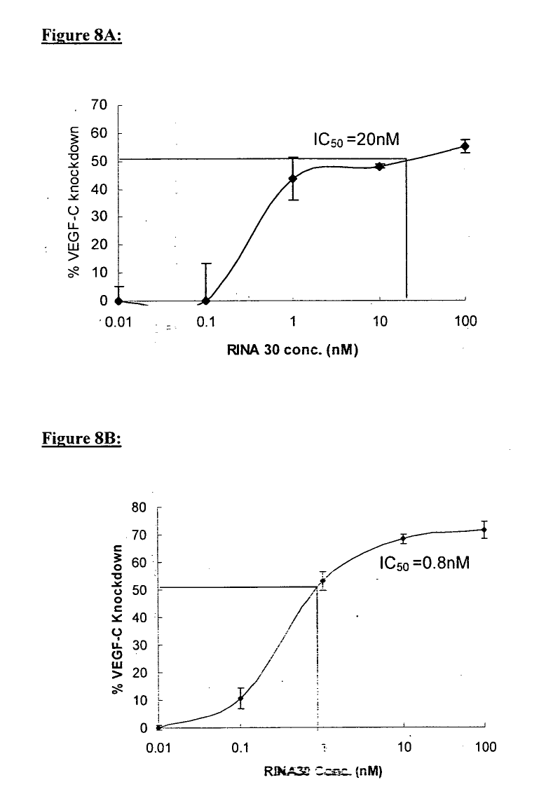

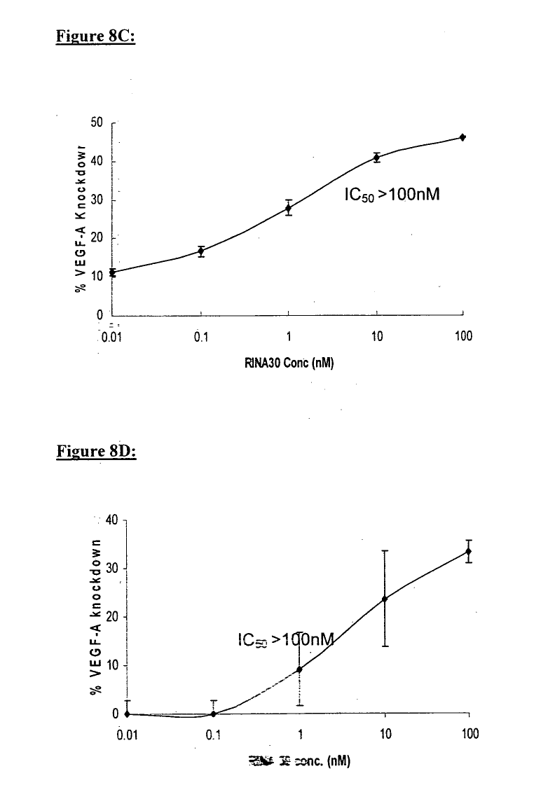

RPE19 and MCF7 cells transfected with RINA 30 showed knockdown of VEGF-C in a dose dependent manner where IC₅₀=20 nM in RPE19 cells, and IC₅₀=0.8 nM in. MCF7 (FIGS. 8A-B). Unlike VEGF-A knockdown causing increased VEGF-C production, VEGF-C knockdown decreased VEGF-A expression (FIGS. 8C-D). Similar results were observed in both hyperglycemic (for RPE19 cells) and hypoxic (for MCF7 cells) conditions respectively (see FIG. 7). Quantitative real time PCR also indicated a decrease in mRNA levels of both VEGF-C and A at the end of 72 h post transfection (Table 5).

### Both VEGF-C And VEGF-A Stimulate Angiogenesis Via RhoA Mediated Pathway

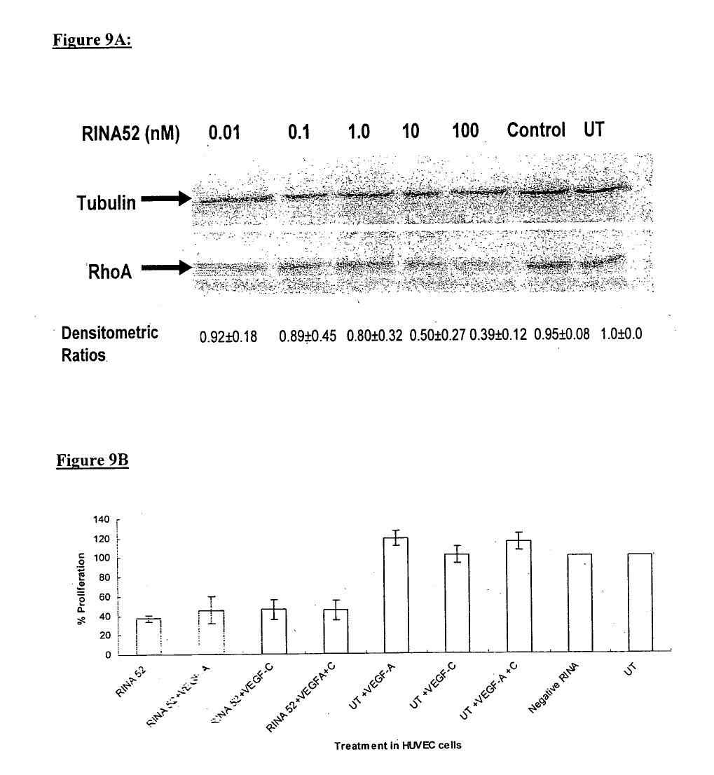

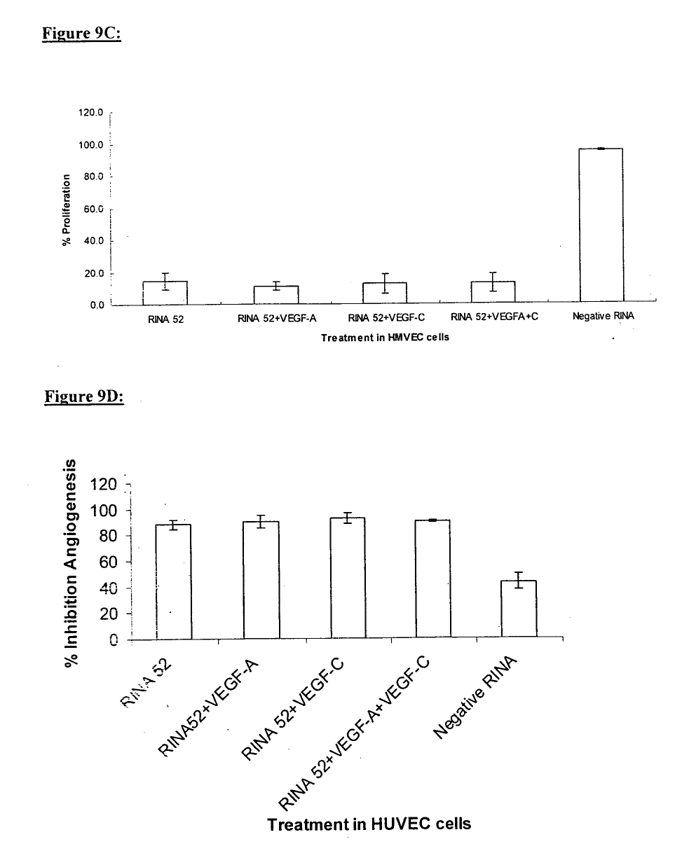

Supplementation of HUVEC cultures with VEGF-A or VEGF-C enhanced HUVEC cell proliferation in a dose-dependent manner (FIG. 12). However, the increase in proliferation was much weaker in case of VEGF-C (only a peak of 12% enhancement). FIG. 9A shows decreased RhoA expression with increased doses of RINA52, reaching maximum knockdown (61%) at 100 nM of RINA52. The cells knocked down for RhoA showed decreased proliferation over negative RINA treated (HUVEC as well as in HMVEC cells) at the end of 72 h post transfection (FIGS. 9B and 9C), indicating that RhoA plays a crucial role in neo-angiogenesis. Supplementation of media with VEGF-A, VEGF-C, or both together (FIGS. 9B and 9C), did not reverse the inhibition of proliferation caused by RhoA knockdown.

Similar results were obtained from tube formation assays, which showed that supplementation of endothelial cells with VEGF-A, VEGF-C, or combination of both, was not able to restore angiogenesis (FIG. 9D) in RhoA knocked down cells. These results indicate that both VEGF-A and VEGF-C induce angiogenesis via a RhoA mediated pathway.

### RhoA Knockdown Decreases Stress Fiber Formation

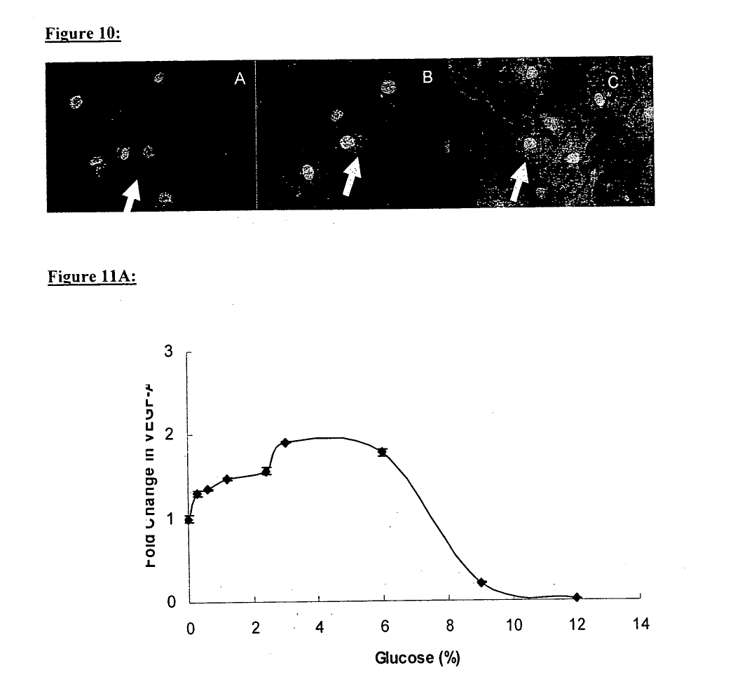

HUVEC cells treated with RINA52 showed a decrease in the number of actin stress 72 h after transfection (FIG. 10). These results indicate that one of the possible mechanisms by which RhoA inhibits angiogenesis is by regulating cytoskeletal proteins such as F-actin to form stress fibers and thus effects the migration, proliferation and contractility of endothelial cells.

### Discussion

The data demonstrates that the knockdown of VEGF-A causes increases VEGF-C expression in both RPE19 and MCF7 cells. Similar results were obtained in hypoxic as well as hyperglycemic conditions (Hyperglycemic conditions have induced expression levels of both VEGF-A and C as shown in FIG. 11), indicating the possibility of similar mechanisms being operated in proliferative retinopathies as well as tumor growth. The increase in expression was observed at the level of both secreted protein and mRNA, indicating that VEGF-A acts at a transcriptional level on VEGF-C.

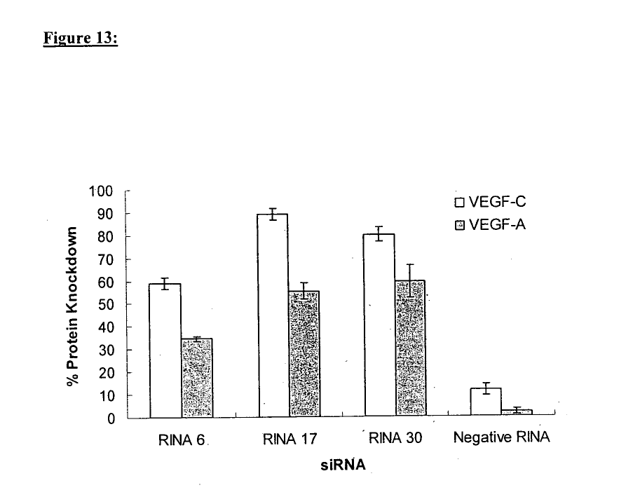

Knockdown of VEGF-C significantly decreased VEGF-A and VEGF-C expression in both RPE19 as well as MCF7 cells, and acted at a transcriptional level on VEGF-A. The decrease in expression levels of VEGF-A was observed not only with RINA30 but also with RINA6 and 17, which target different regions of VEGF-C (FIG. 13). The results obtained in the present study signify that knocking down VEGF-C also regulates neo-angiogenesis being stimulated by VEGF-A by lowering its expression levels. Further, the enhanced expression levels of VEGF-A and C in hypoxic and hyperglycemic conditions could be successfully knocked down by RINA30 indicating the potential of RINA30 in vivo, where hypoxia and/or glycemia may be relevant factors. VEGF-C is a superior target than VEGF-A to inhibit not only lymphangiogenesis but also neo-angiogenesis. In clinical practice, the inhibition of VEGF-A, or its receptor (VEGFR2), have been shown to have therapeutic advantages in controlling neo-angiogenesis in various indications such as cancer and proliferative retinopathies. A number of anti-VEGF small molecule inhibitors (eg: sunitinib) and anti-VEGF monoclonal antibodies (e.g., Avastin) have been developed, which are in clinical use. Their therapeutic efficacy was found to be modest and transitory followed by restoration of neo-angiogenesis and disease progression, however. (Shojaei et al., “Antiangiogenic therapy for cancer: an update,” Cancer J 13, 345-348 (2007); Boneberg et. al., “Angiogenesis and lymphangiogenesis are down regulated in primary breast cancer,” Br J Cancer 101, 605-614 (2009)). The present inventors have shown that the knockdown of VEGF-A leads to enhanced expression of VEGF-C and counteracts the effect of VEGF-A inhibition.The present inventors have also shown that VEGF-C acts through RhoA to mediate angiogenesis, similar to that of VEGF-A. Hence, knockdown of VEGF-C along with RhoA has synergistic effects on inhibiting VEGF-A, and inhibiting angiogenesis. Moreover, the present inventors demonstrate that VEGF-C knockdown results in simultaneous decrease in expression levels of VEGF-A under normaxic, hypoxic, normal and hyperglycemic conditions, and therefore the present invention is applicable under a variety of conditions.

### REFERENCES

1. Folkman J. Angiogenesis in cancer, vascular, rheumatoid and other diseases. Nature Med. 1995; 1: 149-153.

2. Sreekumar P G, Zhou J, Sohn J, Spee C, Ryan S J, Maurer B J, Kannan R, Hinton D R. N-(4-hydroxyphenyl) Retinamide augments laser induced choroidal neovascularization in mice. Investi. Ophthal. & Visual Sci 2008; 49: 1210-1220.

3. Apte R S, Barreiro R A, Duh E, Volpert O, Ferguson T A. Stimulation of neovascularization by the anti-angiogenic factor PEDF. Investi. Ophthal. & Visual Sci. 2004; 45: 4491-4497.

4. Waltman S R, Oestrich C, Krupin T, et. Al Quantitative vitreaous fluorophotometry. A sensitive technique for measuring early breakdown of the blood-tretinal barrier in young diabetic patients. Diabetis 1978; 27: 85-87.

5. Coldwell R B, Slapnick S M, McLaughlin B J. Lanthanum and freeze fracture studies of retinal pigment epithelial cell junctions in the streptozotocin diabetic rat. Curr. Eye. Res 1985; 4: 215-217.

6. Zhao, B, Ma A, Cai J, Boulton M. VEGF-A regulates the expression of VEGF-C in human retinal pigment epithelial cells. Br. J Ophthalmol 2006; 90:1052-1059.

7. Gulec S A, Woltering E A. A new in vitro assay for human tumor angiogenesis: Three dimensional human tumor angiogenesis assay. Ann Surg Oncol, 2004; 11: 99-104.

8. Menakuru S R, Brown N J, Staton C A, Reed M W R. Angiogenesis in pre-malignant conditions. Br J Cancer 2008; 99:1961-1966.

9. Mandriota S J, Jussila L, Jeltsch M, Compagni A, Baetens D, Prevo R, et al Vascular endothelial growth factor—C-mediated lymphangiogenesis promotes tumor metastasis. EMBO J, 2001; 20:672-682.

10. Karpanen T, Egeblad M, Karkkainen M J, Kubo H, Jackson D G, Yla-Herttula S et. al Vascular endothelial growth factor C promotes tumor lymphangiogenesis and intralymphatic tumor growth. Cancer Res. 2001, 88:1786-1790.

11. Skobe M, Hawighorst T, Jackson D G, Prevo R, Janes L, Velasco P. Induction of tumor lymphangiogenesis by VEGF-C promotes breast cancer metastasis. Nat Med 2001, 7: 192-198.

12. Takahashi H, Shibuya M. The vascular endothelial growth factor (VEGF)/VEGF receptor system and its role under physiological and pathological conditions. Clin Sci (Lond). 2005, 109: 227-241.

13. Suarez S C, Fjallman Z, Hofer K B. The role of VEGF receptors in angiogenesis; complex partnerships. Cell Mol Life Sci 2006; 63: 601-6125.

14. Folkman J, Angiogenesis: an organizing principle for drug discovery? Nature Rev. Drug Discov. 2007; 6: 273-286.

15. Ferrara N, Hillan K J, Novotny W. Bevacizumab( Avastin), a humanized anti-VEGF monoclonal antibody for cancer therapy. Biochem. Biophys. Res. Commun. 2005; 333: 328-335.

16. Jain R K. Antiangiogenic therapy for cancer: Current and emerging tconcepts. Oncology (Williston park) 2005, 19: 7-16.

17. Ferrara N, Damico L, Shams N, Lowman H, Kim R. Development of ranibizumab, an anti-vascular endothelial growth factor antigen binding fragment, as therapy for neovascular age-related macular degeneration. Retina, 2006; 26:859-870.

18. Hoang M V, Whelan M C, Senger D R. Rho activity critically and selectively regulates endothelial cell organization during angiogenesis. Proc. Natl. Acad. Sci 2004; 101: 1874-1879.

19. Amerongen G P N, Koolwijk P, Versteilen A, Hinsberg V W M. Involvement of RhoA/Rho kinase signaling in VEGF-induced endothelial cell migration and angiogenesis in vitro. Arterioscler Thromb Vasc Biol 2003, February: DOI:10.1161/01.ATV.0000054198.68894.88.

20. Tammela T, Zarkada G, Wallgard E, Murtomaki A, Suchting S, Wirzenius M et. al. Blocking VEGFR-3 suppresses angiogenic sprouting and vascular network formation. Nature. 2008; doi:10.1038/nature070803.

21. Benest A V, Harper S J, Herttuala S Y, Alitalo K, Bates D O. VEGF-C induced angiogenesis preferentially occurs at a distance from lypmphangiogenesis. Cardiovascular Res. 2008; 78:315-323.

22. Joukov V, Sorsa T, Kumar V, Jeltsch M, Claesson-Welsh L, Cao Y, Saksela O et. al., Proteolytic processing regulates receptor specificity and activity of VEGF-C. EMBO J 1997; 16: 863-868.

23. McColl B K, Baldwin M E, Roufail S, Freeman C, Moritz R L, Simpson R J et. al., Pasmin activates the lymphangiogenic growth factors VEGF-C and VEGF-D. J Exp Med 2003; 198: 863-868.

24. Saaristo A, Tammela T, Farkkila A, Karkkainen M, Suominen E, Herttuala S Y, Alitalo K. Vascular endothelial growth factor-C accelerates diabetic wound healing. Amer. J Pathol 2006; 169: 1080-1087.

25. Berger G, Hanahan D. Modes of resistance to anti angiogenic therapy. Nature Rev. 2008; 8: 592-603.

26. Shojaei F, Ferrara N. Antiangiogenic therapy for cancer: an update. Cancer J 2007; 13: 345-348.

27. Boneberg E M, Legler D F, Hoefer M M, Ohlschlegel C, Steininger H, Fuzesi L et. Al., Angiogenesis and lymphangiogenesis are down regulated in primary breast cancer. Br J Canc 2009; 101: 605-614.

28. Yin L, Morishige K I, Takahashi T, Hashimoto K, Ogata S, Tsutsumi S et. al., Fasudil inhibits vascular endothelial growth factor-induced angiogenesis in vitro and in vivo. Mol Cancer Ther 2007; 6: 1517-1525.

29. Seasholtz T M, Brown J H. Rho signaling in vascular diseases. Mol Interventions 2004; 4: 348-357.

30. Zhao B, Smith G, Cai J, Ma A, Boulton M. Vascular endothelial growth factor C promotes survival of retinalvascular endothelial cells via vascular endothelial growth factor receptor-2. Br J Ophthalmol 2007; 91: 538-545.

All of the compositions and methods disclosed and claimed herein can be made and executed without undue experimentation in light of the present disclosure. While the compositions and methods of this invention have been described in terms of certain embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents that are chemically or physiologically related may be substituted for the agents described herein while the same or similar results would be achieved. All such similar substitutes and modifications apparent to those skilled in the art are deemed to be within the spirit, scope and concept of the invention as defined by the appended claims.

1. A composition comprising:
a first short nucleic acid molecule having 19 to 30 nucleotides that modulates VEGF-C expression, wherein the first short nucleic acid molecule comprises a first nucleotide sequence, wherein a sequence of at least 19 contiguous nucleotides in the first nucleotide sequence is at least 95% complementary to a sequence selected from the group consisting of SEQ ID NO: 1, SEQ ID NO: 2 and SEQ ID NO: 3; and
a second short nucleic acid molecule having 19 to 30 nucleotides that modulates RhoA expression, wherein the second short nucleic acid molecule comprises a second nucleotide sequence, wherein a sequence of at least 19 contiguous nucleotides in the second nucleotide sequence is at least 95% complementary to a sequence selected from the group consisting of SEQ ID NO: 4, SEQ ID NO: 5 and SEQ ID NO: 6.

2. The composition of claim 1, wherein a sequence of at least 19 contiguous nucleotides in the first nucleotide sequence is completely (100%) complementary to a sequence selected from the group consisting of SEQ ID NO: 1, SEQ ID NO: 2 and SEQ ID NO: 3, and wherein a sequence of at least 19 contiguous nucleotides in the second nucleotide sequence is completely (100%) complementary to a sequence selected from the group consisting of SEQ ID NO: 4, SEQ ID NO: 5 and SEQ ID NO: 6.

3. The composition of claim 1, wherein the first short nucleic acid molecule having 19 to 30 nucleotides comprises a siNA selected from the group consisting of:
RINA 6 comprising sense strand SEQ ID NO: 7 and antisense strand SEQ ID NO: 8;
RINA 17 comprising sense strand SEQ ID NO: 9 and antisense strand SEQ ID NO: 10; and
RINA 30 comprising sense strand SEQ ID NO: 11 and antisense strand SEQ ID NO: 12.

4. The composition of claim 1, wherein the second short nucleic acid molecule having 19 to 30 nucleotides comprises a siNA selected from the group consisting of:
RINA 50 comprising sense strand SEQ ID NO: 13 and antisense strand SEQ ID NO: 14;
RINA 51 comprising sense strand SEQ ID NO: 15 and antisense strand SEQ ID NO: 16; and
RINA 52 comprising sense strand SEQ ID NO: 17 and antisense strand SEQ ID NO: 18.

5. The composition of claim 1, wherein the first short nucleic acid molecule having 19 to 30 nucleotides comprises RINA 30 comprising sense strand SEQ ID NO: 11 and antisense strand SEQ ID NO: 12, and the second short nucleic acid molecule having 19 to 30 nucleotides comprises RINA 52 comprising sense strand SEQ ID NO: 17 and antisense strand SEQ ID NO: 18.

6. The composition of claim 1 further comprising a pharmaceutically acceptable carrier.

7. The composition of claim 1, further comprising a lipid, polymer and/or a monoclonal antibody.

8. The composition of claim 1, wherein at least one short nucleic acid molecule is conjugated to cholesterol.

9. The composition of claim 1, wherein the short nucleic acid molecule having 19 to 30 nucleotides is selected from the group consisting of a short interfering nucleic acid (siNA), short interfering RNA (siRNA), double stranded RNA (dsRNA), micro RNA (μRNA), short hairpin RNA (shRNA), and interfering DNA (DNAi) molecules.

10. The composition of claim 9, wherein the short nucleic acid molecule is short interfering RNA (siRNA).

11. A method for reducing or down-regulating VEGF-A secretion from a cell comprising contacting the cell with a short nucleic acid molecule having 19 to 30 nucleotides comprising a sequence of at least 19 contiguous nucleotides that is at least 95% complementary to a sequence of at least 19 contiguous nucleotides within a full length VEGF-C or RhoA gene.

12. The method of claim 11, wherein the short nucleic acid molecule having 19 to 30 comprises a antisense strand consisting of 27 nucleotides, wherein the 27 nucleotide strand sequence is at least 95% complementary to 27 contiguous nucleotides within a full length VEGF-C or RhoA gene.

13. A method for reducing or down-regulating VEGF-A secretion from a cell comprising contacting the cell with a short nucleic acid molecule having 19 to 30 nucleotides comprising a sequence of at least 19 contiguous nucleotides that is completely (100%) complementary to a sequence of at least 19 contiguous nucleotides within a full length VEGF-C or RhoA gene.

14. The method of claim 13, wherein the short nucleic acid molecule having 19 to 30 nucleotides comprises a antisense strand consisting of 27 nucleotides, wherein the 27 nucleotide strand sequence is is completely (100%) complementary to 27 contiguous nucleotides within a full length VEGF-C or RhoA gene.

15. A method for reducing or down-regulating VEGF-A secretion from a cell comprising contacting the cell with a short nucleic acid molecule having 19 to 30 nucleotides comprising a nucleotide sequence that is at least 95% complementary to a sequence selected from the group consisting of SEQ ID NO: 1, SEQ ID NO: 2 and SEQ ID NO: 3.

16. The method of claim 15, wherein the short nucleic acid molecule having 19 to 30 nucleotides comprises a nucleotide sequence that is completely (100%) complementary to a sequence selected from the group consisting of SEQ ID NO: 1, SEQ ID NO: 2 and SEQ ID NO: 3.

17. A method for reducing or down-regulating VEGF-A secretion from a cell comprising contacting the cell with a short nucleic acid molecule having 19 to 30 nucleotides comprising a nucleotide sequence that is at least 95% complementary to a sequence selected from the group consisting of SEQ ID NO: 4, SEQ ID NO: 5 and SEQ ID NO: 6.

18. The method of claim 17, wherein the short nucleic acid molecule having 19 to 30 nucleotides comprises a nucleotide sequence that is completely (100%) complementary to a sequence selected from the group consisting of SEQ ID NO: 4, SEQ ID NO: 5 and SEQ ID NO: 6.

19. The method of claim 11, wherein the short nucleic acid molecule having 19 to 30 nucleotides directly modulates expression of VEGF-C.

20. The method of claim 11, wherein the short nucleic acid molecule having 19 to 30 nucleotides inhibits VEGF-C expression, and inhibits VEGF-A expression or secretion.

21. The method of claim 11, wherein the short nucleic acid molecule having 19 to 30 nucleotides binds to at least one molecule that modulates expression or secretion of VEGF-A.

22. The method of claim 11, wherein the short nucleic acid molecule having 19 to 30 nucleotides is selected from the group consisting of a short interfering nucleic acid (siNA), short interfering RNA (siRNA), double stranded RNA (dsRNA), micro RNA (μRNA), short hairpin RNA (shRNA), and interfering DNA (DNAi) molecules.

23. The method of claim 11, wherein the short nucleic acid molecule having 19 to 30 nucleotides is between 19 to 30 nucleotides, between 25 and 29 nucleotides, or is 27 nucleotides

24. The method of claim 11, wherein the short nucleic acid molecule having 19 to 30 nucleotides comprises a siNA selected from the group consisting of:
RINA 6 comprising sense strand SEQ ID NO: 7 and antisense strand SEQ ID NO: 8;
RINA 17 comprising sense strand SEQ ID NO: 9 and antisense strand SEQ ID NO: 10; and
RINA 30 comprising sense strand SEQ ID NO: 11 and antisense strand SEQ ID NO: 12.

25. The method of claim 11, wherein the short nucleic acid molecule having 19 to 30 nucleotides comprises a siNA selected from the group consisting of:
RINA 50 comprising sense strand SEQ ID NO: 13 and antisense strand SEQ ID NO: 14;
RINA 51 comprising sense strand SEQ ID NO: 15 and antisense strand SEQ ID NO: 16; and
RINA 52 comprising sense strand SEQ ID NO: 17 and antisense strand SEQ ID NO: 18.

26. A method of claim 11, wherein VEGF-A secretion is reduce or down-regulated in a cell after contacting the cell with the short nucleic acid molecule having 19 to 30 nucleotides at a concentration of at least 0.6 nM.

27. A method for reducing or down-regulating VEGF-A secretion from a cell comprising contacting the cell with a first short nucleic acid molecule having 19 to 30 nucleotides and second short nucleic acid molecule having 19 to 30 nucleotides, wherein the first short nucleic acid molecule comprises a sequence of at least 19 contiguous nucleotides that is at least 95% complementary to a sequence of at least 19 contiguous nucleotides within a full length VEGF-C gene, and
wherein the second short nucleic acid molecule comprises a sequence of at least 19 contiguous nucleotides that is at least 95% complementary to a sequence of at least 19 contiguous nucleotides within a full length RhoA gene.

28. The method of claim 27, wherein the first short nucleic acid molecule comprises a sequence of at least 19 contiguous nucleotides that is completely (100%) complementary to a sequence of at least 19 contiguous nucleotides within a full length VEGF-C gene, and wherein the second short nucleic acid molecule comprises a sequence of at least 19 contiguous nucleotides that is completely (100%) complementary to a sequence of at least 19 contiguous nucleotides within a full length RhoA gene.

29. A method for treating a ocular disorder or neovascular disease comprising administering to a subject the composition of claim 1.

30. A method for treating cancer comprising administering to a subject the composition of claim 1.

31. A method for reducing or inhibiting angiogenesis or neoangiogenesis in a tissue comprising contacting the tissue with a first short nucleic acid molecule having 19 to 30 nucleotides and second short nucleic acid molecule having 19 to 30 nucleotides, wherein the first short nucleic acid molecule comprises a sequence of at least 19 contiguous nucleotides that is at least 95% complementary to a sequence of at least 19 contiguous nucleotides within a full length VEGF-C gene, and wherein the second short nucleic acid molecule comprises a sequence of at least 19 contiguous nucleotides that is at least 95% complementary to a sequence of at least 19 contiguous nucleotides within a full length RhoA gene.

32. The method of claim 31, wherein the first short nucleic acid molecule having 19 to 30 nucleotides comprises a sequence of at least 19 contiguous nucleotides that is completely (100%) complementary to a sequence of at least 19 contiguous wherein the second short nucleic acid molecule comprises a sequence of at least 19 contiguous nucleotides that is at least 95% complementary to a sequence of at least 19 contiguous nucleotides within a full length RhoA gene.

33. A method for reducing or inhibiting angiogenesis or neoangiogenesis in a tissue comprising contacting the tissue with the composition of claim 1.

34. A method for reducing or inhibiting ROCK phosphorylation in a cell comprising contacting the cell with a short nucleic acid molecule having 19 to 30 nucleotides comprising a sequence of at least 19 contiguous nucleotides that is at least 95% complementary to a sequence of at least 19 contiguous nucleotides within a full length RhoA gene, wherein phosphorylation of the ROCK is reduced or inhibited, but ROCK expression is not knocked down.

35. The method of claim 34, wherein the short nucleic acid molecule having 19 to 30 nucleotides modulates RhoA expression, and wherein the short nucleic acid molecule comprises a nucleotide at least 19 contiguous nucleotides in the nucleotide sequence is at least 95% complementary to a sequence selected from the group consisting of SEQ ID NO: 4, SEQ ID NO: 5 and SEQ ID NO: 6.

36. The method of claim 34, wherein the short nucleic acid molecule having 19 to 30 nucleotides comprises a siNA selected from the group consisting of:
RINA 50 comprising sense strand SEQ ID NO: 13 and antisense strand SEQ ID NO: 14;
RINA 51 comprising sense strand SEQ ID NO: 15 and antisense strand SEQ ID NO: 16; and
RINA 52 comprising sense strand SEQ ID NO: 17 and antisense strand SEQ ID NO: 18.

37. Use of the composition of claim 1 for the inhibition of angiogenesis or neoangiogenesis.

38. The use of claim 37, wherein the angiogenesis occurs under the conditions of hypoxia or hyperglycemia.

39. The use of claim 38, wherein the angiogenesis is associated with an ocular disorder.

40. The use of claim 39, wherein the ocular disorder is selected from retinopathy and glaucoma.

41. (canceled)
